SEQUENCE LISTING (4 sequences)

SEQ ID NO: 1 - DNA, 20 bp, Artificial Sequence
  synthetic artificial DNA antigen
gtgtgtgtgtgtgtgtgtgt

SEQ ID NO: 2 - DNA, 20 bp, Artificial Sequence
  synthetic artificial DNA antigen
cacacacacacacacacaca

SEQ ID NO: 3 - protein, 30 aa, Artificial Sequence
  peptide linker
GGEGGGEGEEEGGGEGGEGGEEGGGEEDGG

SEQ ID NO: 4 - protein, 9 aa, Artificial Sequence
  Synthetic peptide
VLFGLGFAI

(19) United States
(12) Patent Application Publication
(10) Pub. No.: US 2021/0363220 A1
(43) Pub. Date: Nov. 25, 2021

(54) METHODS AND REAGENTS TO TREAT AUTOIMMUNE DISEASES AND ALLERGY

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 17/344,932

(22) Filed: Jun. 10, 2021

Related U.S. Application Data
(63) Continuation-in-part of application No. 16/029,594, filed on Jul. 7, 2018, now abandoned.
Application No. 16/029,594 is a continuation-in-part of application No. 15/723,173, filed on Oct. 3, 2017, now abandoned.
Application No. 15/723,173 is a continuation of application No. 16/566,716, filed on Sep. 10, 2019, now abandoned.
Application No. 16/566,716 is a continuation-in-part of application No. 16/029,594, filed on Jul. 7, 2018, now abandoned.
(60) Provisional application No. 62/529,476, filed on Jul. 7, 2017.
(60) Provisional application No. 62/404,204, filed on Oct. 5, 2016.
(60) Provisional application No. 62/470,338, filed on Mar. 13, 2017.
(60) Provisional application No. 62/730,523, filed on Sep. 12, 2018.

(51) Int. Cl. C07K 14/74; A61P 37/00

(52) U.S. Cl. CPC C07K 14/70539; A61K 38/00; A61P 37/00

(57) ABSTRACT

Compositions, reagents, formulations and methods to treat disease including autoimmune diseases and allergy are described. The compositions and formulations comprise an antigen causing immune intolerance, an immunosuppressant and a viscosity enhancing agent or a thermal phase changing agent. The reagent is a polymer conjugate comprising an antigen causing immune intolerance and an immunosuppressant conjugated to a polymer.

### CROSS-REFERENCE TO RELATED APPLICATIONS

This application is a continuation-in-part application of U.S. application Ser. No. 16/029,594 filed on Jul. 7, 2018, which claims priority to U.S. provisional patent application Ser. No. 62/529,476 filed on Jul. 7, 2017 and is a continuation-in-part application of U.S. application Ser. No. 15/723,173 filed on Oct. 3, 2017. This application is also a continuation application of U.S. application Ser. No. 16/566,716 filed on Sep. 10, 2019, which claims priority to U.S. provisional patent application Ser. No. 62/730,523 filed on Sep. 12, 2018 and is a continuation-in-part application of U.S. application Ser. No. 16/029,594 filed on Jul. 7, 2018. The entire disclosure of the prior applications is considered to be part of the disclosure of the instant application and is hereby incorporated by reference.

### REFERENCE TO SEQUENCE LISTING

In accordance with MPEP 502.05(L), the present specification makes reference to a Sequence Listing (submitted electronically as a .txt file named “2021_8_ST25.txt” on Aug. 12, 2021. The .txt file was generated on Aug. 12, 2021 and is 1.42 kb in size. The entire contents of the are herein incorporated by reference.

### BACKGROUND OF THE INVENTION

### Field of the Invention

The current invention relates to compositions and reagents to treat disease such as autoimmune disease and allergy. The current invention also discloses methods to treat autoimmune disease and allergy.

### Background Information

Immune responses are necessary for protection against potentially pathogenic microorganisms. However, undesired immune activation can cause injurious processes leading to damage or destruction of one's own tissues. Undesired immune activation occurs, for example, in autoimmune diseases where antibodies and/or T lymphocytes react with self-antigens to the detriment of the body's tissues. This is also the case in allergic reactions characterized by an exaggerated immune response to certain environmental matters and which may result in inflammatory responses leading to tissue destruction. This is also the case in rejection of transplanted organs which is significantly mediated by alloreactive T cells present in the host which recognize donor alloantigens or xenoantigens. Immune tolerance is the acquired lack of specific immune responsiveness to an antigen to which an immune response would normally occur. Typically, to induce tolerance, there must be an exposure to a tolerizing antigen, which results in the death or functional inactivation of certain lymphocytes. This process generally accounts for tolerance to self-antigens, or self-tolerance. Immunosuppressive agents are useful in prevention or reduction of undesired immune responses, e.g., in treating patients with autoimmune diseases or with allogeneic transplants. Conventional strategies for generating immunosuppression associated with an undesired immune response are based on broad-acting immunosuppressive drugs. Additionally, in order to maintain immunosuppression, immunosuppressant drug therapy is generally a life-long proposition. Unfortunately, the use of broad-acting immunosuppressants is associated with a risk of severe side effects, such as tumors, infections, nephrotoxicity and metabolic disorders. Accordingly, new immunosuppressant therapies would be beneficial.

### BRIEF DESCRIPTION OF THE DRAWINGS

FIG. 1 shows example of general structure of antigen-drug conjugate

FIG. 2 shows example of general structure of antigen-alpha gal conjugate

FIG. 3 shows an example of antigen-alpha gal conjugate for SLE treatment

FIG. 4 shows examples of 3 different formats of the antigen-drug conjugate.

FIG. 5 shows examples of an antigen-sialic acid rich polymer conjugate to treat autoimmune disease or allergy or to induce immune tolerance.

FIG. 6 shows examples of the conjugate containing antigen and sialic acid/Siglec ligand.

FIG. 7 shows schematic example of the structure of the microsphere based agent to induce immune tolerance and treating autoimmune diseases or allergy.

FIG. 8 shows different formats of polymer carrier conjugated with antigen, Siglec ligand and other immunosuppressant; and both Siglec ligand and other immunosuppressant conjugated to the antigen.

FIG. 9 shows examples of Siglec ligand-antigen conjugate for systemic lupus erythematosus treatment.

FIG. 10 shows schematic example of multiple antigens and immunosuppressants with linkers to form a linear polymer.

FIG. 11 shows exemplary scheme of antigen containing polymer conjugated to a nano or micro particle encapsulating immune suppressant (drug).

FIG. 12 shows exemplary scheme of multiple antigens with linkers to form a linear polymer.

FIG. 13 shows exemplary scheme of multiple antigen conjugated to a polymer carrier backbone.

FIG. 14 shows exemplary scheme of antigen containing polymer conjugated to a nano or micro particle.

FIG. 15 shows exemplary scheme of coating additional TB regulatory cell stimulating molecule/cytokine to pMHC-NP/MP.

FIG. 16 shows exemplary scheme of multiple pMHC is conjugated to or expressed in a polymer instead of being coated on a particle.

### DESCRIPTION OF THE INVENTIONS AND THE PREFERRED EMBODIMENT

Previous U.S. application Ser. Nos. 15/723,173, 16/380,951 and 16/029,594 by the current inventor disclose methods, agents, devices and compositions to treat autoimmune diseases and allergy and to prevent antigen specific antibody generation including anti-drug antibody generation. The agents in the previous US applications include antigen-drug conjugate such as antigen-immunosuppressant conjugate. The agents and compositions can also be a mixture of antigen and immunosuppressant molecule or their conjugate. They can be in the form of linear polymer, micro particle, nano particle, liposome, implant or a transdermal drug delivery system such as a transdermal patch. Examples of the antigen include B cell antigen, T cell antigen in MHC-peptide complex form or the antigen peptide (or its derivative) that can bind with MHC. A carrier system can be used for the previous and current applications to construct the conjugate. For example, the liposome or microparticle or nanoparticle can be used as a carrier. The antigen can be immobilized on the surface of the liposome or particles and the effector molecule (e.g. alpha gal, rhamnose, immune suppression cytokine, tregitope peptide, toxin, siRNA or miRNA or the like, immunosuppressant, antisense molecule) can be either encapsulated inside or co-immobilized on the surface of liposome or particles. The carrier can also be a linear or branched polymer such as dextran, hyaluronic acid, heparin, chondroitin sulfate and polypeptide. Both antigen and the effector molecule (such as immunosuppressant) can be conjugated to the polymer. They can be given to the subject in need to treat autoimmune diseases and allergy or inhibit anti-drug antibody production or induce antigen specific immune tolerance by administering to the subject said conjugate (e.g. subcutaneous or intravenous injection or applied to the skin such as the skin of upper arm). Additional details can be found in the previous disclosures.

In one aspect, the current invention discloses a transdermal drug delivery system such as a transdermal patch to treat conditions selected from autoimmune disease, allergy and anti-drug antibody comprising an antigen causing said condition and an immunosuppressant. The current invention also discloses a method to treat autoimmune disease or allergy or inhibit anti-drug antibody production or induce antigen specific immune tolerance in a subject by administering to the subject a said transdermal drug delivery system on the skin. Example of immunosuppressant can be selected from rapamycin, fujimycin and methotrexate.

In another aspect, the current invention discloses a conjugate in linear polymer form or particle form to treat conditions selected from autoimmune disease, allergy and anti-drug antibody or to inhibit anti-drug antibody production or to induce antigen specific immune tolerance comprising an antigen causing the condition, a first immunosuppressant and an optional second immunosuppressant. The antigen can be B cell antigen, T cell antigen in MHC-peptide complex form or the antigen peptide (or its derivative) that can bind with MHC. The first immunosuppressant is selected from Siglec ligand such as sialic acid or poly sialic acid. Example of second immunosuppressant is selected from rapamycin, fujimycin, methotrexate and PD-L1. The current invention also discloses a method to treat autoimmune disease or allergy or inhibit anti-drug antibody production or induce antigen specific immune tolerance in a subject by administering to the subject said conjugate (e.g. subcutaneous or intravenous injection).

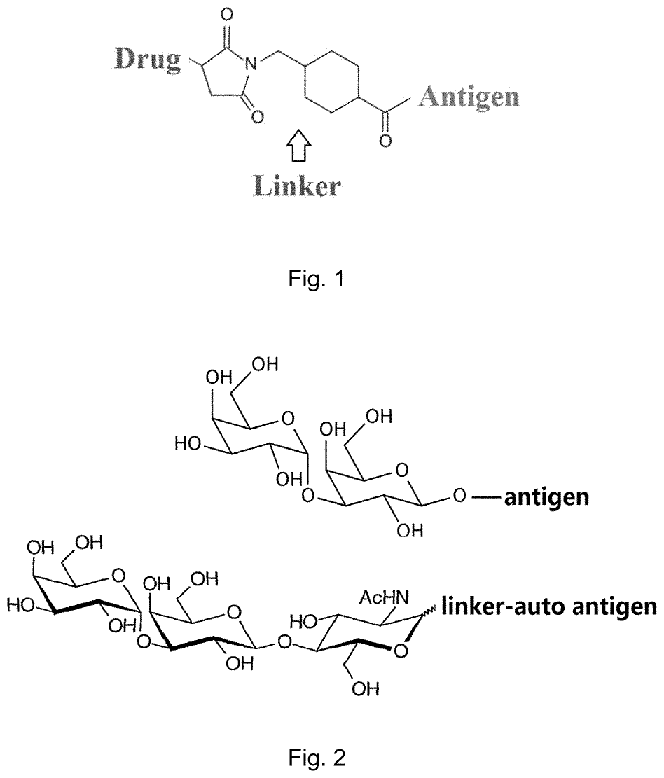

Previous U.S. application Ser. No. 15/723,173 by the inventor discloses antigen-drug conjugate to treat autoimmune diseases and allergy or inhibit anti-drug antibody production or induce antigen specific immune tolerance with general structure as shown in FIG. 1. Autoantibody against DNA is a key pathogenic factor in SLE, DNA coated affinity column is clinically used to remove these Ab from patient blood (hemopurification) as an effective SLE treatment. Antigen-drug conjugate can be used for SLE treatment. DNA-linker-mertansine (DNA sequence adopted from Abetimus, linker/toxin adopted from Kadcyla, linker can be optimized for B/T cells) is an example of ADC for SLE treatment. The DNA sequence used are the complex formed with GTGTGTGTGTGTGTGTGTGT (SEQ ID NO: 1) and CACACACACACACACACACA (SEQ ID NO: 2). Single strand DNA antigen can also be used to inactivate autoantibody generating cells specific to single strand DNA. It will selectively inactivate the specific B cell clone producing auto antibody autoantibody against DNA, treat the disease from the source. It can be prepared easily with solid phase synthesis. It can be intravenously injected to the patient having SLE to treat it. Companion test will be performed to increase the efficacy. Patient will be treated with hemopurification to remove the anti-DNA antibody before the first dose ADC administration for better therapeutical index.

Instead of epitope (antigen)-toxin described, epitope (antigen)-alpha gal (e.g. galactose-alpha-1,3-galactose) can also be used instead, which utilize the endogenous anti-gal antibody to inactivate the B cell clone or T cell clone that can selectively bind with the epitope (antigen). The alpha gal can be readily adopted from US patent application U.S. Ser. No. 12/450,384 and other publication. Epitope (antigen)-alpha gal conjugate design has the formula: alpha galactosyl-(optional linker)-epitope (antigen), which will allow the T cell/B cell specific to the epitope (antigen) bind with endogenous anti-Gal antibody and therefore be eliminated/inactivated due to the bound antibody. Examples of its structure are shown in FIG. 2.

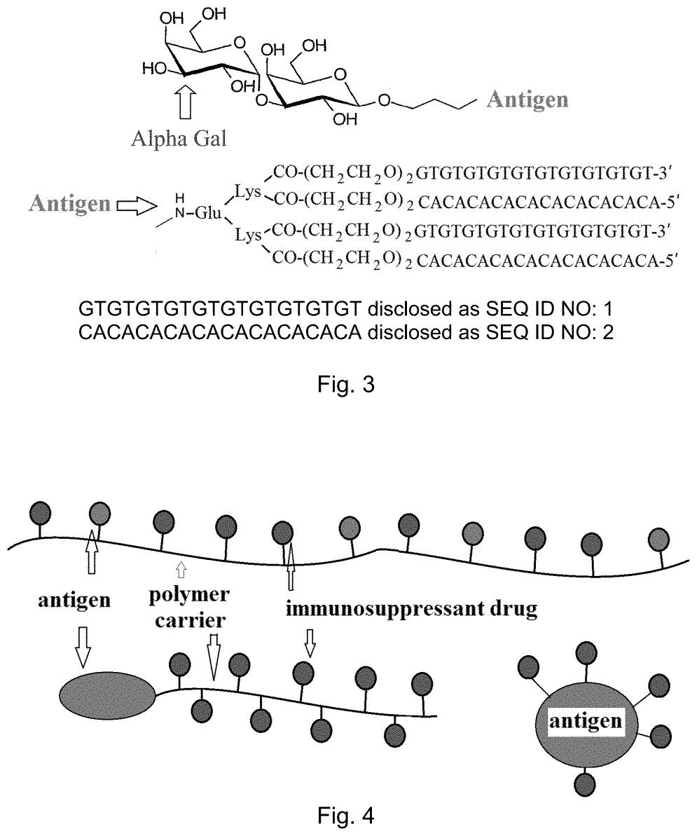

An example reagent that can selectively inactivate B cells producing autoantibody against DNA is shown in FIG. 3, this drug can be used to treat lupus. The patient can receive 500 mg˜1 g of the said conjugate as weekly i.v. injection to treat his lupus until symptom disappears.

In another aspect, the current invention discloses a method to treat a condition selected from autoimmune disease, allergy and anti-drug antibody. The method involves isolating the immune cells specific to disease related pMHC multimer, in vitro expansion and then transferring the cells to the subject in need to treat said disease.

In another aspect, the current invention and previous applications from the current inventor disclose methods, compositions and reagents to treat autoimmune diseases and allergy or to inhibit anti-drug antibody production or to induce antigen specific immune tolerance by applying the combination of antigen and immunosuppressive agent/drug either as a physical mixture or as synthetic conjugate or as nano/micro/macro particles or implant or liposome to the subject/patient in need. The term nano/micro particle means the particle is in either nanometer or micrometer range of size (diameter). For example, the nano/micro particle can be in the size range of 50 nm-100 μm. The macro particle can be in the size range of 100 μm-10 mm. The particles can be made of biodegradable materials such as PLGA. A physical mixture means that the mixture of antigen and immunosuppressive agent are simply mechanically mixed (e.g. by stirring or blending) together in their original form (e.g. liquid or solid form such as powder or particles) without any additional process (e.g. by mixing them in their original form together), or further size reducing process is applied after the mechanical mixing (e.g. crashing, grinding, mulling or homogenizing), or dispersed or dissolved separately in same or different type of liquid and then mix, or co-dispersed in liquid, or co-dissolved in solvent (e.g. water), and optional drying process (e.g. spray drying or lyophilization) can be applied with optional further size reducing process. List of exemplary immunosuppressive drugs can be found in the article page for immunosuppressive drug in Wikipedia. The immunosuppressive agent/drug (immunosuppressants) suitable for the current application include but are not limited to, statins; mTOR inhibitors, such as rapamycin or a rapamycin analog; TGF-β signaling agents; TGF-β receptor agonists; TLR (toll like receptor) inhibitors; pattern recognition receptor inhibitors; NOD-like receptors (NLR) inhibitors; RIG-I-like receptors inhibitors; NOD2 inhibitors; histone deacetylase inhibitors such as trichostatin A; corticosteroids; inhibitors of mitochondrial function such as rotenone; P38 inhibitors; NF-κβ inhibitors such as 6Bio, dexamethasone, TCPA-1, IKK VII; adenosine receptor agonists; prostaglandin E2 agonists (PGE2) such as misoprostol; phosphodiesterase inhibitors such as phosphodiesterase 4 inhibitor (PDE4), for example rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide, Siglec ligand such as sialic acid and its derivative including poly sialic acid and sialic acid-lipid conjugate. In embodiments, the immunosuppressant may comprise any of the agents provided herein. The immunosuppressant can be a compound that directly provides the immunosuppressive (e.g., tolerogenic) effect on APCs or it can be a compound that provides the immunosuppressive (e.g., tolerogenic) effect indirectly (i.e., after being processed in some way after administration). Immunosuppressants, therefore, include prodrug forms of any of the compounds provided herein.

The immunosuppressant also includes heme oxygenase-1 (HO-1) inducer such as cobalt protoporphyrin (CoPP), protoporphyrin IX containing a ferric iron ion (heme B) with a chloride ligand (hemin), hematin, iron protoporphyrin or heme degradation products as well as those described in PCT/EP2015/074819. Siglecs (sialic acid-binding immunoglobulin-type lectins) ligand such as sialic acid or its derivatives is also another type of immunosuppressant that can be used in current invention. PD-L1 is also another type of immunosuppressant that can be used in current invention. PD-L1 can effectively inhibit cytotoxic T cell. Fragment or mimic or derivative of PD-L1 that can bind with PD-1 can also be used instead. Other inhibitory ligands that can bind with inhibitory checkpoint receptor (e.g. A2AR, BTLA, CTLA-4, CD 47, KIR, LAG3, TIM-3, VISTA and etc.) such as B7-H3, B7-H4 can also be used instead of PD-L1. Molecule that can promote T/B reg expansion (e.g. cytokine that can stimulate T/B reg expansion such as IL-2 and TGF-β) is also another type of immunosuppressant. Different immunosuppressant can be used as a mixture and be used in combination in the current invention.

The immunosuppressant can be a compound that directly provides the immunosuppressive (e.g., tolerogenic) effect on APCs or it can be a compound that provides the immunosuppressive (e.g., tolerogenic) effect indirectly (i.e., after being processed in some way after administration). Immunosuppressants, therefore, include prodrug forms of any of the compounds provided herein.

Immunosuppressant also include nucleic acids that encode the peptides, polypeptides or proteins provided herein that result in an immunosuppressive (e.g. tolerogenic) immune response. In embodiments, therefore, the immunosuppressant is a nucleic acid that encodes a peptide, polypeptide or protein that results in an immunosuppressive (e.g., tolerogenic) immune response. The nucleic acid can be coupled to synthetic nanocarrier. The nucleic acid may be DNA or RNA, such as mRNA. In embodiments, the inventive compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector.

In some embodiments, the immunosuppressants provided herein are coupled to synthetic nanocarriers or microcarriers. In preferable embodiments, the immunosuppressant is an element that is in addition to the material that makes up the structure of the synthetic nanocarrier or microcarrier. For example, in one embodiment, where the synthetic nanocarrier or microcarrier is made up of one or more polymers, the immunosuppressant is a compound that is in addition and coupled to the one or more polymers. As another example, in one embodiment, where the synthetic nanocarrier or microcarrier is made up of one or more lipids, the immunosuppressant is again in addition and coupled to the one or more lipids. In embodiments, such as where the material of the synthetic nanocarrier or microcarrier also results in an immunosuppressive (e.g., tolerogenic) effect, the immunosuppressant is an element present in addition to the material of the synthetic nanocarrier or microcarrier that results in an immunosuppressive (e.g., tolerogenic) effect.

Other exemplary immunosuppressants include, but are not limited, small molecule drugs, natural products, antibodies (e.g., antibodies against CD20, CD3, CD4), biologics-based drugs, carbohydrate-based drugs, nanoparticles, liposomes, RNAi, antisense nucleic acids, aptamers, methotrexate, NSAIDs; fingolimod; natalizumab; alemtuzumab; anti-CD16, anti-CD3; tacrolimus (FK506), etc. Further immunosuppressants, are known to those of skill in the art, and the invention is not limited in this respect. Additional immunosuppressants can be found in patent and patent application serial numbers U.S. Ser. No. 13/880,778, U.S. Ser. No. 14/934,135, CA 2910579, U.S. Ser. No. 13/084,662, U.S. Ser. No. 14/269,048, U.S. Pat. No. 8,652,487 and other patent application filed by Selecta Biosciences.

Additional immunosuppressants can be found in patent application serial numbers WO2012054920A2, WO2016073799A1, WO2012149393 A3, WO2014179771A1, PCT/US2012/035405, US20110262491, patent serial number U.S. Pat. No. 8,652,487 and other patent application filed by Selecta Biosciences. Selecta's publications disclose synthetic nanocarrier methods, and related compositions, comprising B cell and/or MHC Class II-restricted epitopes and immunosuppressants in order to generate tolerogenic immune responses. In their disclosure, the antigen/epitope is conjugated to the nanocarrier and immunosuppressants is coupled to the nanocarrier.

An alternative method and composition are to use nano/micro particle having antigen/epitope non-covalently adsorbed to its surface and immunosuppressant encapsulated within. The nano/micro particles can be made of biodegradable materials such as PLGA. These kinds of nano/micro particles (e.g. 10 nm˜10 nm of diameter in size) can be given to the patient in need as injection or inhaler or applied topically to induce immune tolerance. The encapsulation of immunosuppressant is well known to the skilled in the art and can be adopted from related publications readily. The surface of the nano/micro particles can have charged groups such as amino or carboxyl group to increase the binding of antigen/epitope to its surface; it can also have a hydrophobic surface to allow binding antigen/epitope via hydrophobic interaction; or the combination of them. Introducing charged groups to the surface can be done by using surface modification or using amine or carboxyl group containing molecules to prepare the nano/micro particles. The antigen/epitope can also be conjugated with a lipid molecule such as fatty acid or cholesterol to increase its binding to nano/micro particles. The adsorption of antigen/epitope to the nano/micro particle surface can be done by incubating antigen/epitope with the nano/micro particle (e.g. 4 degree overnight in aqueous solution buffer such as 1×PBS) and then removing the unbound antigen/epitope (e.g. washing the nano/micro particle with aqueous buffer several times, similar to ELISA plate coating procedure). In one example, 50 nm˜200 nm size PLGA nanoparticle encapsulated with 10% by weight of rapamycin is prepared according to the literature. Next the PLGA nanoparticle is mixed with OVA (10 mg/mL) at 4 C overnight to generate the OVA (ovalbumin) coated particle. The particle is washed 3 times with PBS to remove unbound OVA. In another example, rapamycin is dissolved in DMSO at 50 mg/ml. A total of 50 μL rapamycin is added to 1 ml PLGA (5 mg/ml) dissolved in dichloromethane. Next the mixture is homogenized with 0.4 ml 5% OVA solution for 10 min using ultrasonication. The o/w emulsion is added to 2.1 ml of a 5% w/v solution of PVA to evaporate the organic solvent for 4 h at room temperature. OVA coated nanoparticles containing rapamycin are obtained after centrifugation at 3,500 g for 20 min. Additional washing step can be performed to obtain unbound OVA free particles. This OVA coated particle can be given to the target in need to induce OVA immune tolerance, using the similar protocol described in the publications (e.g. those from Selecta Bio). For example, 5 mg˜50 mg of the particle can be injected to a patient with OVA intolerance weekly for 3 times to induce OVA tolerance as subcutaneous or intravenous injection or intralymphatic injection. The OVA can be replaced with other antigen/epitope molecule to induce corresponding immune tolerance. In another sample, lipophilic carboxylic acid or lipophilic amine or anionic detergent or cationic detergent (e.g. fatty acid such as caprylic acid, lauric acid; or cationic lipid such as DOTMA, DOTAP, cholesterylamine) can be added to the PLGA to prepare PLGA particle having surface charge. In one example, rapamycin is dissolved in DMSO at 50 mg/ml with lauric acid at 10 mg/mL. A total of 50 μL rapamycin/lauric acid is added to 1 ml PLGA (5 mg/ml PLGA) dissolved in dichloromethane. Next the mixture is homogenized with 0.1 ml 2% caprylic acid solution for 10 min using ultrasonication. The o/w emulsion is evaporated to remove the organic solvent for 4 h at room temperature. The resulting PLGA particle is washed 3 times with PBS and then incubated with OVA to prepare OVA bound particles. In one example, 10 mg˜100 mg of the particle can be injected to a patient with OVA intolerance bi-weekly for 3 times to induce OVA tolerance as subcutaneous or intravenous injection or intralymphatic injection.

Furthermore, antigen/epitope can also be encapsulated within the nano/micro particle besides being conjugated or adsorbed to its surface. The preparation of antigen/epitope encapsulation is well known to the skilled in the art and can be adopted from related publications readily, e.g. using a double emulsion water/oil/water system.

US patent application serial number US20130287729A1 disclosed antigen-specific, tolerance-inducing microparticles and uses thereof. It disclosed microparticle (0.5 μm-10.0 μm in size) for targeting an antigen-presenting immune cell of interest and for inducing antigen-specific immune tolerance, wherein the microparticle comprises an antigen and a therapeutic agent wherein the therapeutic agent is an immunomodulatory agent, an immunosuppressive tolerogenic agent, or an agent that recruits the antigen-presenting immune cell of interest, wherein the surface of the microparticle comprises a ligand that targets the antigen-presenting immune cell of interest and the microparticle is made of biodegradable material. A further improvement of this method and composition is to use a nano/micro particle having the size of 50 nm˜5 μm, preferably made of biodegradable materials. In some embodiments, the surface of the nano/micro particle is coated with Fc portion of an antibody or a full antibody with its Fc portion facing outside. This will bind with the FcR to facilitate APC uptake. In other embodiments, the surface of the nano/micro particle needs not to have a ligand that targets the antigen-presenting immune cell. In some embodiments, it can have antigen/epitope coated on its surface. The inner part of the nano/micro particle contains immunosuppressive agent listed in the current application and optionally antigen/epitope, e.g. by encapsulation. The preparation method is well known to the skilled in the art and can be adopted from related publications readily. For example, 5 mg˜50 mg of the above particle containing gluten and rapamycin can be injected to a patient with gluten intolerance weekly for 3 times to induce gluten tolerance as subcutaneous or intravenous injection or intralymphatic injection.

US patent application 20160338953A1 disclosed a liposome-based immunotherapy. It provided a liposome encapsulating an autoantigen, wherein the liposome has a size comprised from 500 to 15000 nm and the liposome membrane comprises phosphatidylserine (PS) in an amount comprised from 10 to 40% by weight with respect to the total membrane liposomal composition. Pharmaceutical or veterinary compositions comprising a therapeutically effective amount of said liposome were also provided. Further, it provided liposomes and pharmaceutical or veterinary compositions as defined above for use as a medicament, particularly for the treatment of autoimmune diseases. Finally, it provided liposomes and pharmaceutical or veterinary compositions as defined above for use in the restoration of tolerance in a patient suffering from an autoimmune disease. The current invention also discloses antigen-specific, tolerance-inducing liposome and uses thereof. The liposome contains immunosuppressive agent listed in the current application (and optionally antigen/epitope molecule) inside by encapsulation. Optionally the surface of the liposome can also have antigen/epitope coated. It can be given to the patient in need as injection or inhaler or applied topically to induce immune tolerance. The lipid used for liposome can include but not limited to phosphatidylserine at 10 to 40% by weight of the membrane. It can also use non-phosphatidylserine lipid to prepare the membrane. The antigen/epitope can also be conjugated with a lipid type molecule such as fatty acid or phospholipid or cholesterol derivative to allow it to be inserted to the liposome membrane. Suitable liposome can have a size between 50 nm˜20 μm. The preparation method and the protocol of its use are well known to the skilled in the art and can be adopted from related publications readily such as those in patent application No. US20160338953. Example of the lipid molecule suitable for the current invention to prepare liposome includes but is not limited to phospholipid glycerolipid, glycerophospholipid, sphingolipid, ceramide, glycerophosphoethanolamine, sterol or steroid. These lipid molecules can also be used to prepare the antigen/epitope-lipid conjugate. Membrane anchoring peptide-antigen/epitope conjugate can also be used instead of antigen/epitope-lipid conjugate. In addition, other molecule that can promote TB reg expansion (e.g. IL-2 and/or TGF-β and PD-L1) can also be coated/conjugated to and/or encapsulated within the liposome and nano/micro particle. For example, 5 mg˜50 mg of the said liposome containing egg white antigen such as ovomucoid and rapamycin can be injected to a patient with egg white intolerance weekly for 3 times to induce egg white tolerance as subcutaneous or intravenous injection or intralymphatic injection at inguinal lymph node.

Previous U.S. application Ser. Nos. 15/723,173, 16/380,951 and 16/029,594 by the current inventor disclose methods and reagents to treat autoimmune disease, allergy and to induce immune tolerance for specific antigens. They disclosed novel reagents and compositions comprising antigen and immunosuppressant. Those novel reagents and formulations can be given as either subcutaneous injection or intramuscular injections or intradermal injections or intravenous injection at pharmaceutical effective amount to treat autoimmune disease or allergy or inhibit anti-drug antibody production or induce antigen specific immune tolerance in a subject. Furthermore, for the same indication those reagents and compositions can also be injected into lymph node (e.g. inguinal lymph node) instead. Intralymphatic allergen administration is known and the same procedure can be readily adopted for the current invention. The reagents and formulations disclosed as prior arts in the patent applications PCT/US2018041170 and U.S. Ser. No. 16/029,594 can also be used as intralymphatic injection. Molecule that can promote T/B reg expansion and/or inhibit harmful auto reactive T/B cell (e.g. IL-2, TGF-β, PD-L1, IL-15, IFN-y, IL-10, IL-21, IL-27, IL-2/anti-IL-2 antibody complexes or their mimics or derivatives such as a pegylated IL-2 NKTR-358) can also be co-injected or included in the formulation to be injected intralymphaticly. The reagents and formulations in said previous applications and current invention by the current inventor contains disease specific antigen such as B cell antigen, T cell antigen in MHC-peptide complex form or the antigen epitope, mimotope, peptide (or its derivative) of T cell antigen that can bind with MHC to form the MHC-peptide complex. Instated of using antigen directly in the said reagent or formulation, nucleic acid encoding these antigen/epitope can also be used instead such as mRNA encoding them. The mRNA can be in a delivery system such as liposome or lipid vector and can also be modified to improve the target expression using well know methods and protocol. In some embodiments, the amount of the reagent or formulation injected into lymph node is between 0.01 mg 50 mg with injection volume between 0.1 ml to 1 ml per lymph node such as 1 mg weekly or monthly for 3 weeks or 3 months to induce the antigen specific immune tolerance.

Previous U.S. application Ser. Nos. 15/723,173, 16/380,951 and 16/029,594 by the current inventor and current invention disclose methods and reagents to treat autoimmune diseases and allergy by applying the mixture of antigen and immunosuppressive agent topically to the object/patient in need. It can also be used to inhibit the generation of anti-drug antibody when the antigen is the drug (e.g. a protein drug) or its epitope. It will induce immune tolerance for the antigen. Examples of the formulation suitable for the current application include solid form such as powder, gel, lotion, ointment, solution, spray, suppository, lozenge, tablet and patch that can be topically applied to the skin or mucosa. The term topical drug delivery includes drug delivery route other than injection. It includes applying drug to skin or mucosa. It includes intranasal delivery, rectal delivery, sublingual delivery and oral mucosa delivery. The immunosuppressive agent can be in the form of active agent, prodrug form, microparticle or nanoparticle form or liposome form. The antigen can be either B cell antigen/epitope or T cell antigen/epitope (e.g. MHC-peptide complex or conjugate; or the peptide antigen that can bind with MHC) or their combination. The combination can be either B cell antigen/epitope with T cell antigen/epitope; or the combination of several different B cell antigen/epitope and/or several different T cell antigen/epitope targeting the same disease or different diseases. The use of peptide antigen (T cell epitope) that can bind with MHC to form MHC-peptide complex in vivo (T cell antigen) instead of the peptide-MHC complex reduce the size and molecular weight, therefore improve the transdermal delivery. Examples of them can be found in the current application and related publications and patent applications.

In some embodiments, the method is to use a patch containing both antigen/allergen and immune-suppressive drug (the drug listed above such as rapamycin or fujimycin or methotrexate or sialic acid or its derivative or high affinity Siglec binder or their combination). The sialic acid can be either free sialic acid or sialic acid ester or sialic acid-lipid conjugate form. For example, sialic acid can be conjugated to cholesterol to form an ester bond using the —COOH of sialic acid with the —OH of the cholesterol. This conjugate will have better transdermal and cell membrane permeation capability. The fatty acid can also be conjugated with sialic acid's —OH to form the conjugate. These conjugates will work as immune-suppressive drug after being transdermally delivered. Examples of high affinity Siglec ligands can be found in US patent serial number U.S. Pat. No. 8,357,671.

The transdermal or transmucosal delivery of both antigen and immunosuppressive drug will induce immune tolerance via DC cells in the skin or mucosa. The skin may be exfoliated to remove stratum corneum layer to increase drug delivery or using a microneedle system. This would be a much easier strategy for food allergy and autoimmune disease treatment than injection. The skin may be intact or may be exfoliated to remove stratum corneum layer to increase drug delivery. Microneedle system can also be used to the skin. The micro needle in the microneedle system can be made of biodegradable material such as PLGA encapsulating antigen and immunosuppressant. Alternatively, a biodegradable implant encapsulating antigen and immunosuppressant can also be used. The size of the implant can be bigger than 10 μm in diameter, preferably >100 μm, if the implant is a macroparticle. For example, a 2 mm (length)×0.3 mm (diameter) rod made with PLGA containing 3 mg rapamycin and 1 mg gliadin can be used as an implant underneath the skin to treat gluten intolerance. Other implant format can also be used such as NanoPortal Capsule from Nanoprecision Medical and Medici Drug Delivery System from Intarcia, as long as they can deliver the antigen and immunosuppressant simultaneously.

DBV Technologies and other groups (e.g. those described in Epicutaneous Immunotherapy for Aeroallergen and Food Allergy DOI: 10.1007/s40521-013-0003-8) are using skin patch containing allergen to treat allergy by inducing tolerance for the antigen (allergen). The topically patch or other formulation can be readily adopted for the current application. For example, the topical applied formulation such as patch described in U.S. Ser. No. 15/135,914, U.S. Pat. No. 6,676,961, U.S. Ser. No. 15/111,204, U.S. Pat. No. 8,932,596B2, U.S. Ser. No. 15/184,933A1 and U.S. Pat. No. 8,202,533B2 can be adopted for the current application by adding additional immune suppressive drug in the patch (e.g. 0.1 mg˜20 mg of rapamycin or fujimycin or 1 mg˜100 mg methotrexate or their derivatives or prodrug) as well as those commercial available patch (e.g. VIASKIN® MILK and VIASKIN® PEANUT). The administration method can be essentially the same as the prior arts except the patch contains immunosuppressants. Additional transdermal enhancer (e.g. DMSO, azone, fatty acid, hyaluronic acid etc., which can be found in the publication readily as well as their suitable amount) can be added to the patch or applied to the skin before applying the patch. Examples of transdermal enhancing agent can be added include DMSO (e.g. 10˜300 mg/patch), azone (e.g. 1%˜10% of total drug weight), surfactant, fatty acid (e.g. 1%˜10% oleic acid). The skin stratum corneum can also be removed with exfoliation or other means to enhance the transdermal delivery. In one example, the patch contains 500 μg˜10 mg gluten (e.g. G5004 gluten from wheat, Sigma) and 0.1 mg˜10 mg of rapamycin or 1 mg˜50 mg methotrexate. For example, antigen such as gluten and immunosuppressant such as rapamycin and/or methotrexate can be in powder form, which can be simply mixed together physically, they can also be codissolved and then dried and then placed in the patch. For example, 10 mg gluten powder and 1 mg of rapamycin powder are blended and then homogenized with a grinder, and then applied to the surface of the skin contact side of a 5×5 cm² dermal patch. In another example, 10 mg gluten and 1 mg of rapamycin are mixed in 10 mL water containing 30 mg sucrose vigorously for 10 min and then lyophilized, and then the dry mixture is applied to the surface of the skin contact side of a 5×5 cm² dermal patch. In another example, 10 mg gluten and 1 mg of rapamycin are dissolved in 5 mL 25% EtOH water solution and then vacuum dried, and then the dry mixture is placed to the surface of the skin contact side of a 3×3 cm² dermal patch. In another example, 10 mg gluten and 1 mg of rapamycin are dissolved in 5 mL 1% SDS water solution and then vacuum dried, and then the dry mixture is placed to the surface of the skin contact side of a 3×2 cm² dermal patch. In another example, the patch contains 5 mg gluten (e.g. G5004 gluten from wheat, Sigma) and 0.1˜5 mg of rapamycin or 50 mg methotrexate and optionally additional 30 mg azone. In another example, the patch contains 5 mg gluten (e.g. G5004 gluten from wheat, Sigma) and 100 mg of sialic acid or sialic acid-cholesterol conjugate or 10 mg methotrexate. This can be used to induce gluten tolerance and treat gluten intolerance. The gluten can be replaced with gliadin instead. In embodiments, the gluten containing patch can be applied to forearm daily for 1-5 weeks. The gluten in the above examples can be replaced with egg white protein such as 5˜10 mg of ovomucoid (Gal d 1) or 5˜10 mg ovalbumin (Gal d 2) or their combination with optional 5˜10 mg ovotransferrin (Gal d 3) and 5˜10 mg lysozyme (Gal d 4) to treat egg white allergy. In another example, the antigen is peanut antigen ara h2 200 μg and 2 mg of rapamycin is in the patch to treat peanut allergy. In one example, peanut antigen ara h2 200 μg, 2 mg of rapamycin and 50 mg sucrose is dissolved in water and then lyophilized and then placed in the patch. In one example, peanut antigen ara h2 200 μg, 0.5 mg of rapamycin, 50 mg SDS and 50 mg sucrose is dissolved in water and then lyophilized and then placed in the patch. In one example, peanut antigen ara h2 200 μg, 2 mg of rapamycin, 100 mg DMSO and 50 mg sucrose is dissolved in water and then lyophilized and then placed in the patch. In another example, the antigen is the double strand DNA (1 mg˜10 mg) in the previous figures to treat lupus and the drug is 3 mg of rapamycin or fujimycin or temsirolimus. In another example, the nasal spray contains 1 mg gluten (e.g. G5004 from Sigma, gluten from wheat) and 1 mg of rapamycin or 10 mg methotrexate in a suitable form for each spray. In another example, the sublingual lozenge contains 50 mg gluten (e.g. G5004 from Sigma, gluten from wheat) and 1 mg of rapamycin or 20 mg methotrexate. In another example, the gel contains 50 mg gluten (e.g. G5004 gluten from wheat, Sigma) and 2 mg of rapamycin or 20 mg methotrexate in each 1 ml of gel. The immunosuppressant drug or both the immunosuppressant drug and the antigen can be either in the form of powder or gel or semiliquid or in the form of liposome (e.g. 100 nm˜5 nm diameter) or in a nano/micro particle (e.g. 100 nm˜1 μm) or being conjugated to a dendrimer or linear polymer (e.g. couple to poly acrylic acid or poly Sialic acid via ester bond to form a polymer based prodrug with MW=5KD˜500KD).

Other pharmaceutically acceptable amount of antigen and immunosuppressant can also be used in the patch, as long as it can produce satisfactory biological and therapeutical (e.g. immune tolerance) effect, which can be determined experimentally by screening and testing with well-known protocol and methods.

Suitable antigen can be either B cell antigen/epitope or T cell antigen/epitope (e.g. MHC-peptide complex or conjugate; or the peptide antigen that can bind with MHC) including their coding nucleic acid or their combination. Examples of them can be found in the current application and said previous applications by the same inventor and related publications and patent applications.

The transdermal delivery of both antigen and immunosuppressive drug will be uptaken by APC in the skin, induce/activate tolerogenic dendritic cell and Treg/Breg, inhibit B cell activation/antibody production, germinal center formation and antigen-specific hypersensitivity reactions, resulting in long term antigen specific immune tolerance.

A skin patch (also called transdermal patch) is a medicated adhesive patch or attachable patch that is placed on the skin to deliver a specific dose of medication through the skin and into the bloodstream. A wide variety of pharmaceuticals are now available in transdermal patch form.

There are several main types of skin/transdermal patches. The single-layer drug-in-adhesive type is that the adhesive layer of this system also contains the drug. In this type of patch, the adhesive layer not only serves to adhere the various layers together, along with the entire system to the skin, but is also responsible for the releasing of the drug. The adhesive layer is surrounded by a temporary liner and a backing. The multi-layer drug-in-adhesive type is similar to the single-layer system; the multi-layer system is different, however, in that it adds another layer of drug-in-adhesive, usually separated by a membrane (but not in all cases). One of the layers is for immediate release of the drug and other layer is for control release of drug from the reservoir. This patch also has a temporary liner-layer and a permanent backing. The drug release from this depends on membrane permeability and diffusion of drug molecules. The reservoir type is unlike the single-layer and multi-layer drug-in-adhesive systems; the reservoir transdermal system has a separate drug layer. The drug layer can be a liquid or gel or powder compartment containing a drug solution or suspension or powder separated by the adhesive layer. This patch is also backed by the backing layer. In this type of system the rate of release is zero order. The matrix type has a drug layer of a solid or semisolid matrix containing a drug solution or suspension or solid layer such as powder or film. The adhesive layer in this patch surrounds the drug layer, partially overlaying it. In some embodiments, the reservoir type and the matrix type can be used for current invention.

In one example, antigen and immunosuppressant loaded matrix-type transdermal patch is prepared by using solvent casting method. A petri dish with a total area of 50 cm² is used. Antigen and immunosuppressant are dissolved in 5 mL of water, methanol (1:1) solution and mixed until clear solution is obtained. 200 mg polyethylene glycol 400 is used as plasticizer and optional 100 mg propylene glycol or oleic acid or tween 80 is used as permeation enhancer, together with 100 mg sucrose they are added to the antigen/immunosuppressant solution. The resulted uniform solution is cast on the petri dish, which is lubricated with glycerin and lyophilized or dried at room temperature for 24 h. Next the dried patch is placed on a cellulose acetate membrane used as backing membrane. In another example, weighed amount of PVA (polyvinyl alcohol) 2.5% w/v is added to a distilled water and a homogenous solution is made by constant stirring and intermittent heating at 60° C. for a few seconds and poured into glass molds already wrapped with aluminum foil around open ends and are kept for drying at 60° C. for 6 h, forming a smooth, uniform, and transparent backing membrane. Backing membrane is used as a support for antigen and immunosuppressant containing matrix.

In some embodiments, the skin patch device used in the method of the invention preferably comprises a backing, the periphery of said backing being adapted to create with the skin a hermetically closed chamber. This backing bears on its skin facing side within the chamber the composition used to decrease the skin reactivity. Preferably, the periphery of the backing has adhesive properties and forms an airtight joint to create with the skin a hermetically closed chamber.

In a particular embodiment, the composition allergens and immunosuppressants are maintained on the backing by means of electrostatic and/or Van der Waals forces. This embodiment is particularly suited where the composition allergens are in solid or dry form (e.g., particles), although it may also be used, indirectly, where the allergens are in a liquid form. Within the context of the present invention, the term “electrostatic force” generally designates any non-covalent force involving electric charges. The term Van der Waals forces designates non-covalent forces created between the surface of the backing and the solid allergen, and may be of three kinds: permanent dipoles forces, induced dipoles forces, and London-Van der Waals forces. Electrostatic forces and Van der Waals forces may act separately or together. In this respect, in a preferred embodiment, the patch device comprises an electrostatic backing. As used herein, the expression “electrostatic backing” denotes any backing made of a material capable of accumulating electrostatic charges and/or generating Van der Waals forces, for example, by rubbing, heating or ionization, and of conserving such charges. The electrostatic backing typically includes a surface with space charges, which may be dispersed uniformly or not. The charges that appear on one side or the other of the surface of the backing may be positive or negative, depending on the material constituting said backing, and on the method used to create the charges. In all cases, the positive or negative charges distributed over the surface of the backing cause forces of attraction on conducting or non-conducting materials, thereby allowing to maintain the allergen and immunosuppressant. The particles also may be ionized, thereby causing the same type of electrostatic forces of attraction between the particles and the backing. Examples of materials suitable to provide electrostatic backings are glass or a polymer chosen from the group comprising cellulose plastics (CA, CP), polyethylene (PE), polyethylen terephtalate (PET), polyvinyl chlorides (PVCs), polypropylenes, polystyrenes, polycarbonates, polyacrylics, in particular poly(methyl methacrylate) (PMMA) and fluoropolymers (PTFE for example). The foregoing list is in no way limiting.

The back of the backing may be covered with a label which may be peeled off just before application. This label makes it possible, for instance, to store the composition allergen in the dark when the backing is at least partially translucent. The intensity of the force between a surface and a particle can be enhanced or lowered by the presence of a thin water film due to the presence of moisture. Generally, the patch is made and kept in a dry place. The moisture shall be low enough to allow the active ingredient to be conserved. The moisture rate can be regulated in order to get the maximum adhesion forces. As discussed above, the use of an electrostatic backing is particularly advantageous where the allergen is in a dry form, e.g., in the form of particles. Furthermore, the particle size may be adjusted by the skilled person to improve the efficiency of electrostatic and/or Van der Waals forces, to maintain particles on the support.

In a specific embodiment, the patch comprises a polymeric or metal or metal coated polymeric backing and the particles of composition allergens are maintained on the backing essentially by means of Van der Waals forces. Preferably, to maintain particles on the support by Van der Waals forces, the average size of the particles is lower than 60 micrometer. In another embodiment, the allergens are maintained on the backing by means of an adhesive coating on the backing. The backing can be completely covered with adhesive material or only in part. Different occlusive backings can be used such as polyethylene or PET films coated with aluminum, or PE, PVC, or PET foams with an adhesive layer (acrylic, silicone, etc.). Examples of patch devices for use in the present invention are disclosed in patent application serial number U.S. Ser. No. 11/915,926 or U.S. Pat. No. 7,635,488.

Other examples are disclosed in patent application serial number U.S. Ser. No. 13/230,689, which also discloses a spray-drying process to load the substance in particulate form on the backing of a patch device. An electrospray device uses high voltage to disperse a liquid in the fine aerosol. Allergens and immunosuppressants dissolved in a solvent are then pulverized on the patch backing where the solvent evaporates, leaving allergens and immunosuppressants in particles form. The solvent may be, for instance, water or ethanol, according to the desired evaporation time. Other solvents may be chosen by the skilled person. This type of process to apply substances on patch backing allows nano-sized and micro-sized particles with a regular and uniform repartition of particles on the backing. This technique is adapted to any type of patch such as patch with backing comprising insulating polymer, doped polymer or polymer recovered with conductive layer. Preferably, the backing comprises a conductive material.

In another embodiment, the periphery of the backing is covered with a dry hydrophilic polymer, capable of forming an adhesive hydrogel film by contact with the moisturized skin (as described in U.S. Ser. No. 12/680,893). In this embodiment, the skin has to be moisturized before the application of the patch. When the hydrogel comes into contact with the moisturized skin, the polymer particles absorb the liquid and become adhesive, thereby creating a hermetically closed chamber when the patch is applied on the skin. Examples of such hydrogels include polyvinylpyrolidone, sodium polyacrylate, copolymer of methyl vinyl ether and maleic anhydride.

In another particular embodiment, the liquid composition allergen and immunosuppressant is held on the support of the patch in a reservoir of absorbent material. The composition may consist in an allergen+immunosuppressant solution or in a dispersion of the mixture, for example in glycerine. The adsorbent material can be made, for example, of cellulose acetate.

The backing may be rigid or flexible, may or may not be hydrophilic, and may or may not be translucent, depending on the constituent material. In the case of glass, the support may be made break-resistant by bonding a sheet of plastic to the glass. In one embodiment, the backing of the patch contains a transparent zone allowing directly observing and controlling the inflammatory reaction, without necessarily having to remove the patch. Suitable transparent materials include polyethylene film, polyester (polyethylene-terephtalate) film, polycarbonate and every transparent or translucent biocompatible film or material.

Current invention also discloses methods and reagents to treat autoimmune diseases and allergy or to inhibit anti-drug antibody production or to induce antigen specific immune tolerance by applying the mixture of said antigen and said immunosuppressive agent/drug as injection to the object/patient in need. The injection can be given as either subcutaneous injection or intramuscular injections or intradermal injections or intralymphatic injection. The injection contains a viscosity enhancing agent to increase its viscosity after being injected, which acts as a sustained release formulation of both antigen and immunosuppressive agent. Molecule that can promote TB reg expansion (e.g. IL-2 and/or TGF-β and/or PD-L1) can also be added into the injection in combination with other immunosuppressive agent. Antigen and immunosuppressive agent can be either in free molecule form or in nano/micro particle from including liposome form. In certain embodiments, the injection has a viscosity greater than 10,000 cps at room temperature. In certain embodiments, the injection has a viscosity greater than 100,000 cps at room temperature. In certain embodiments, the injection has a viscosity greater than 5,000,000 cps at room temperature. In certain embodiments, the injection has a viscosity of 11,000,000 cps at room temperature. Example of the viscosity enhancing agent can be found readily from known pharmaceutical acceptable excipient such as hyaluronic acid, starch and carbomer. In some embodiments, the viscosity enhancing agent is biodegradable. In one example, a viscous injection contains 5 mg/mL gluten (e.g. G5004 gluten from wheat, Sigma) and 2 mg/mL of rapamycin or 50 mg/mL methotrexate and suitable amount of hyaluronic acid (e.g. 50 mg/mL) to reach a viscosity of 5,000,000 cps with optional 1 mg/mL IL-2. The hyaluronic acid can be crosslinked to extend their in vivo half-life. The injection formulation can also be a thermal phase changing formulation. Thermal phase changing formulation is a formulation that change its phase from liquid at low temperature or room temperature (25° C.) to semisolid/gel when temperature increases to body temperature (37° C.), which can use poloxamer as excipient. A thermal phase changing injectable formulation containing both antigen and immunosuppressive agent can be given as either subcutaneous injection or intramuscular injections or intradermal injections to induce antigen specific immune tolerance and treat corresponding autoimmune diseases or allergy. It has low viscosity at low or room temperature but high viscosity at body temperature. The preparation of this kind of thermal phase changing injectable formulation can be adopted from related publications readily by the skilled in the art. For example, a composition of a thermal phase changing injectable formulation is 15 mg/mL gluten (e.g. G5004 gluten from wheat, Sigma), and 3 mg/mL rapamycin in 25% (w/w) Poloxamer-407 pH=7 solution, which can be injected to a patient with gluten intolerance 1 mL bi-weekly for 3 times to induce gluten tolerance as subcutaneous or intravenous injection or intralymphatic injection. The gluten in the above examples can be replaced with egg white protein such as 5˜10 mg of ovomucoid (Gal d 1) or 5˜10 mg ovalbumin (Gal d 2) or their combination with optional 5˜10 mg ovotransferrin (Gal d 3) and 5˜10 mg lysozyme (Gal d 4) to induce tolerance to egg.

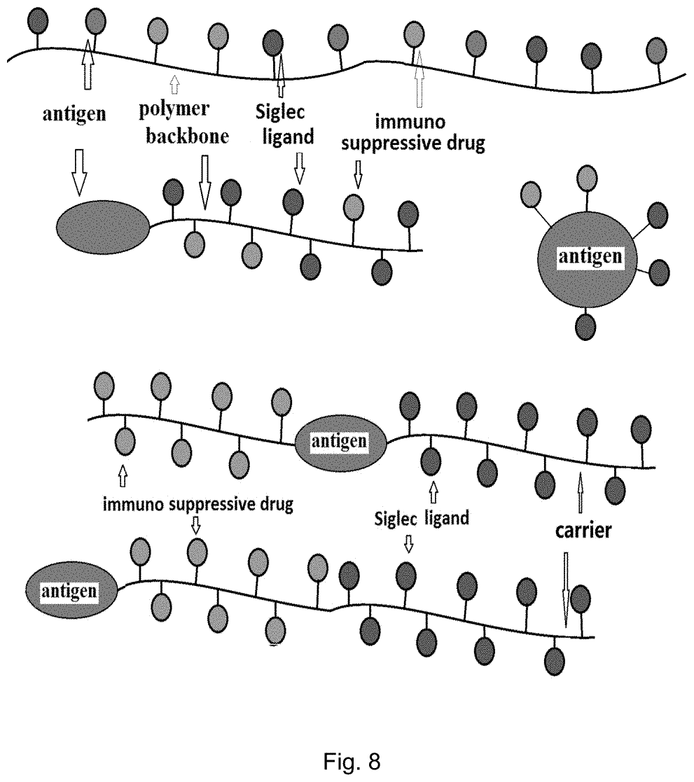

The immunosuppressive agent can also be conjugated to carbohydrate polymer or other bio compatible polymer (e.g. dextran or heparin or hyaluronic acid or polypeptide) to form prodrug as described in U.S. application Ser. Nos. 15/723,173, 16/380,951 and 16/029,594. The novel prodrugs can be in the form of carbohydrate (or other polymer) drug conjugate in which the drug is conjugated to the carbohydrate (or other polymer) with cleavable linkage. More than one drugs can be conjugated to the polymer backbone. Suitable carbohydrate includes sialic acid containing polymer, hyaluronic acid, chondroitin sulfate, dextran, carboxyl dextran, cellulose, carboxyl cellulose and their derivatives. In some embodiments, preferably the carbohydrate is selected from sialic acid containing polymer, hyaluronic acid, starch, dextran, and chondroitin sulfate. The sialic acid containing polymer suitable for the current invention include poly sialic acid formed by sialic acid monomer connected with α2,3 or α2,6 or α2,8 or α2,9 linkage or their combination. It also includes graft polymer or branched polymer containing sialic acid. It can also be a linear polymer backbone (e.g. dextran or synthetic polymer such as PVA, PAM. Furthermore, the immune suppressive drug can also be directly conjugated to antigen or conjugated to the antigen via a linker or carrier and used in the patch. The carrier can be a polymer. For example, the poly sialic acid-rapamycin in FIG. 8 of U.S. application Ser. No. 15/723,173 can be used to conjugate to the protein's lysine with EDC coupling (e.g. gluten or antibody drug or gliadin or is peanut antigen protein ara h2) and be used in the patch (e.g. 100 μg˜15 mg) instead of the mixture of antigen and drug.

When liposome is used, either the drug or both the antigen and immunosuppressive drug can be encapsulated in the liposome. Dendritic cell is abundant in skin, adding DC regulating drug with antigen/allergen in a patch can be effective to induce tolerance. Besides being applied topically, the mixture or conjugate can also be injected or taken orally to induce immune tolerance and to treat autoimmune disease/allergy.

As previously described, those reagents and compositions can be given as either subcutaneous injection or intramuscular injections or intradermal injections or intravenous injection or intralymphatic at pharmaceutical effective amount to treat autoimmune disease or allergy or inhibit anti-drug antibody production or induce antigen specific immune tolerance in a subject.

The topical formulation or implant of the current and previous invention by the current inventors can contain either antigen+drug or antigen-drug conjugate or encapsulated antigen/drug (e.g. in microsphere or nano particle or liposome) or their combinations. The antigen can be either in the form of crude antigen (e.g. peanut extract, gluten, egg white powder) or purified antigen (e.g. peanut antigen protein ara h2, gliadin) or antigen-drug conjugate or encapsulated antigen (e.g. in microsphere or liposome) or their mixture.

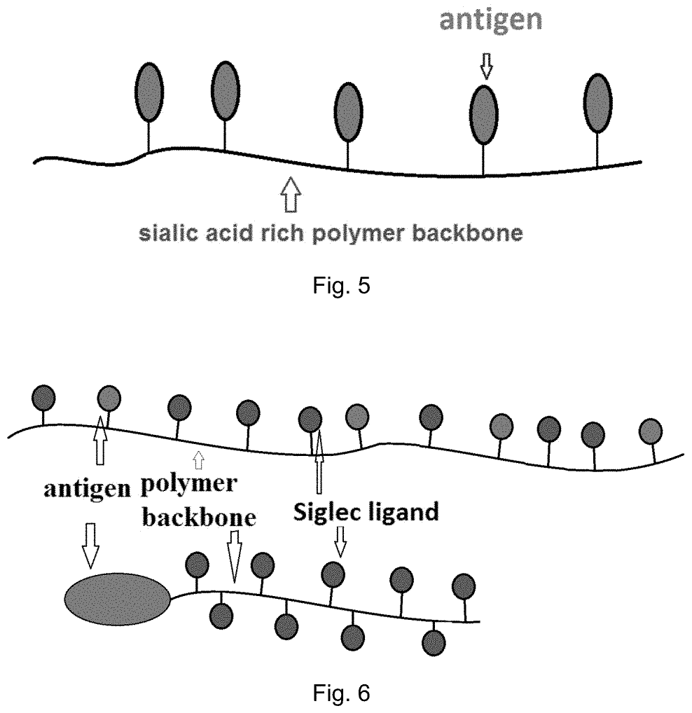

In another format, as shown in FIG. 5, the epitope (antigen)-sialic acid rich polymer conjugate or epitope (antigen)-Siglec ligand rich polymer conjugate can be used to treat autoimmune disease or allergy or to induce immune tolerance, which can be either injected or implanted (being encapsulated inside the implant) or applied topically. The antigen/epitope can be either B cell antigen or T cell antigen or their combination. For example, the lysine group of the antigen can be used to conjugate to the —COOH group of the sialic acid with well-known EDC coupling method. The pharmaceutically acceptable amount of conjugate can also be used, as long as it can produce satisfactory therapeutic (e.g. immune tolerance) effect, which can be determined experimentally by screening and testing with well-known protocol.

The term sialic acid rich polymer means a polymer having multiple sialic acids or Siglec ligand conjugated to its back bone. The back bone can be a branched or linear polymer or dendrimer such as synthetic polymer PVA, PAA, polyamine, or nature polymer such as polysialic acid, carbohydrate. The sialic acid or sialic acid containing fragments or Siglec ligands are conjugated to the polymer back bone. Sialic acid polymer contains either α2,3 or α2,6 or α2,8 sialoside or sialic acid or their derivatives (e.g. those described in J Immunol. 2006 Sep. 1; 177(5):2994-3003, US patent application U.S. Pat. No. 9,522,183 and US Patent U.S. Pat. No. 8,357,671) that can bind with Siglec. The oligo/poly sialic acid with α2,8 linkage backbone itself is also a sialic acid rich polymer. The sialic acid rich polymer can also contains the mixture of different sialoside, sialic acid and/or their derivatives on its backbone. The liposome having sialic acid or sialoside attached on its surface can also be regarded as a sialic acid rich polymer (e.g. those described in US patent U.S. Pat. No. 9,522,183).

There are many sialic acid/Siglec ligand rich polymer suitable for the current application can be readily found in the literature, for example, those described in J Immunol. 2006 Sep. 1; 177(5):2994-3003, Nat Chem Biol. 2014 January; 10(1):69-75, J Am Chem Soc. 2013 Dec. 11; 135(49):18280-18283, J. Immunol. 2014 Nov. 1; 193(9): 4312-21, J Allergy Clin Immunol. 2017 January; 139(1):366-369.e2, Angew Chem Int Ed Engl. 2015 Dec. 21; 54(52):15782-8, Proc Natl Acad Sci USA. 2009 Feb. 24; 106(8):2500-5, J Exp Med. 2010 Jan. 18; 207(1):173-87, J Immunol. 2013 Aug. 15; 191(4):1724-31, Proc Natl Acad Sci U SA. 2016 Sep. 13; 113(37):10304-9, J Clin Invest. 2013 July; 123(7):3074-83, Proc Natl Acad Sci USA. 2016 Mar. 22; 113(12):3329-34, Patent application U.S. Pat. No. 9,180,182 and US Patent U.S. Pat. No. 9,552,183. These sialic acid/Siglec ligand rich polymers can be readily adopted for the current inventions. In some embodiments each polymer is conjugated with more than 10 copies of antigen.

Using epitope (antigen)-sialic acid rich polymer conjugate, the antigen will bind with the auto immune T cell or B cell clones, which will guide the conjugated sialic acid rich polymer to inactivate these antigen specific auto immune T cell or B cell clones selectively.

FIG. 6 shows examples of the conjugate containing sialic acid/Siglec ligand suitable for the current inventions. Optional linkers can be added between the antigen and the polymer and/or between Siglec ligand and the polymer.

When liposome expressing both antigen and Siglec ligand is used (e.g. those described in the current invention and those in J Clin Invest. 2013 July; 123(7):3074-83, J Immunol. 2013 Aug. 15; 191(4):1724-31 and US patent U.S. Pat. No. 9,552,183), the liposome can further encapsulate immunosuppressive drug such as rapamycin. For example, each liposome particle can contain pharmaceutical effective amount of rapamycin (e.g. 1%˜50% liposome weight of rapamycin). This will further increase the efficacy to induce immune tolerance and treating autoimmune diseases/allergy.

Another format suitable for the current application is to use microsphere. The term microsphere includes particles from nano meter size to micrometers (e.g. 50 nm˜50 μm in diameter). Preferably the microsphere is biodegradable (e.g. made of biodegradable polymer such as poly(lactidecoglycolide) (PLGA)), the microsphere can further encapsulate immune suppressive drug such as rapamycin (e.g. 1%˜80% weight of the microsphere).

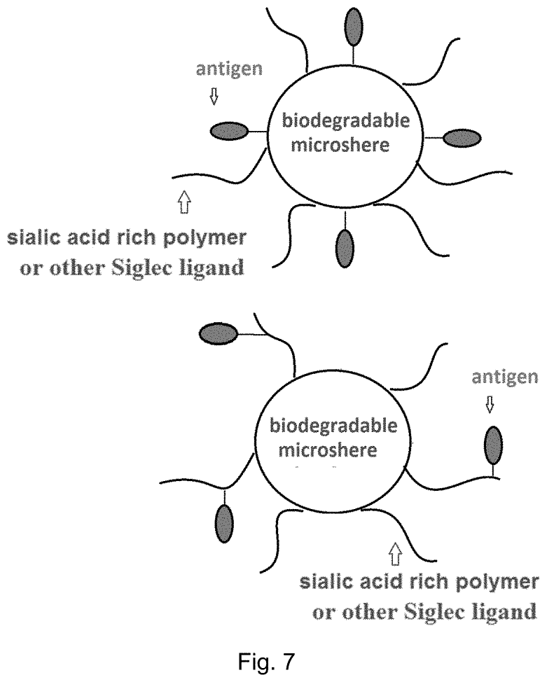

FIG. 7 shows schematic examples of the structure of the microsphere based agent to induce immune tolerance and treating autoimmune diseases/allergy. For example, the microsphere can be biodegradable synthetic polymer such as PLGA. Immune-suppressive drug such as rapamycin (e.g. 1%˜80% weight of the microsphere) is encapsulated. The size of the microsphere is 3 μm or 300 nm. Sialic acid rich polymer or other Siglec ligand is conjugated to the surface of the microsphere directly or with a linker, antigen is also conjugated to the surface of the microsphere directly or with a linker. Alternatively, the sialic acid rich polymer is conjugated to the surface of the microsphere directly or with a linker and the antigen is conjugated to the sialic acid rich polymer. The antigen can also be encapsulated in the microsphere as well. Alternatively, the drug (immunosuppressant) can be conjugated to the surface of the microsphere or conjugated to the sialic acid rich polymer instead of being encapsulated. Examples of microsphere suitable for the current application can be readily adopted from the disclosure in the publications such as those in patent application serial numbers U.S. Ser. No. 13/880,778, U.S. Ser. No. 14/934,135, CA 2910579, U.S. Ser. No. 13/084,662 and US patent U.S. Pat. No. 8,652,487 and other patent application filed by Selecta Biosciences. It can be used to treat autoimmune disease or allergy or to induce immune tolerance, which can be either injected or implanted (being encapsulated inside the implant) or applied topically to the patient. The pharmaceutically acceptable amount of these types of conjugate can also be used, as long as it can produce satisfactory therapeutical (e.g. immune tolerance) effect, which can be determined experimentally by screening and testing with well-known protocol.

They can be used to treat autoimmune disease or allergy or to induce immune tolerance caused by the antigen used to construct these conjugates, which can be either injected or implanted (being encapsulated inside the implant) or applied topically to the subject in need. The pharmaceutically acceptable amount of conjugate in pharmaceutically acceptable formulation can be used, as long as it can produce satisfactory therapeutical (e.g. immune tolerance) effect, which can be determined experimentally by screening and testing with well-known protocol. This method can be used to treat antigen specific autoimmune disease or allergy.

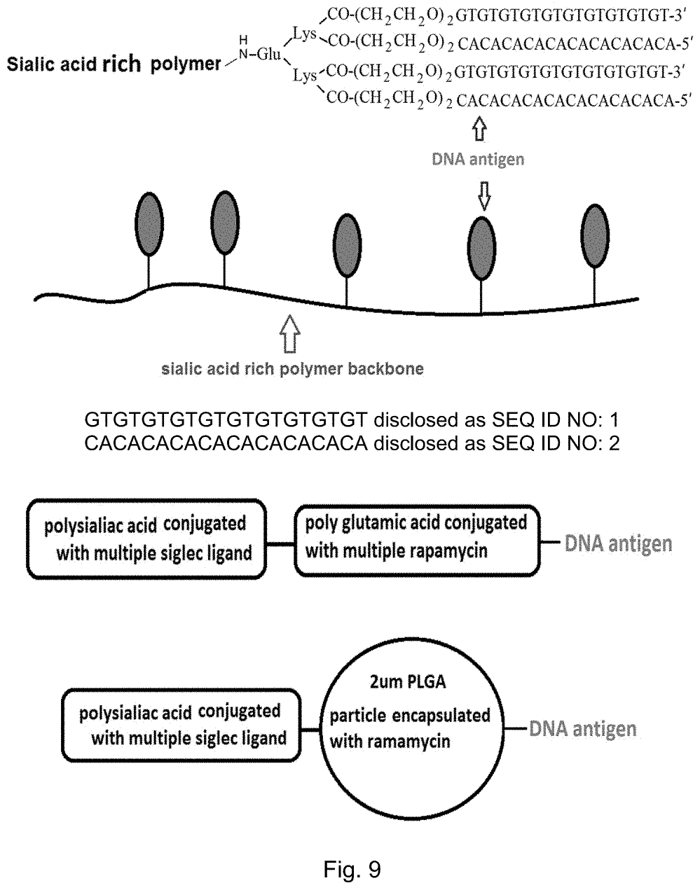

Another format suitable for the current application is to use polymer carrier conjugated with antigen, Siglec ligand and/or other immunosuppressant, which is shown in the FIG. 8. Alternatively, both Siglec ligand and other immunosuppressant can be conjugated to the antigen. FIG. 9 shows different formats suitable for the current invention. The polymer conjugated with multiple antigen (e.g. 1-100), multiple Siglec ligands (e.g. 5˜500 copies) and multiple copies of other immunosuppressant is essentially the previous described polymer conjugated with antigen and Siglec ligand, which is further conjugated with multiple immunosuppressant molecules (e.g. 5˜500 molecules). Alternatively the polymer conjugated with multiple immunosuppressant molecules and multiple Siglec ligands can be conjugated to one antigen molecule. Alternatively, multiple immunosuppressant molecules and multiple Siglec ligands can be conjugated to one antigen molecule directly or with linker but without polymer carrier. Alternatively, one or more polymer conjugated with multiple immunosuppressant molecules and one or more multiple polymer conjugated with Siglec ligands can be conjugated to one antigen molecule. Alternatively, one or more polymer conjugated with multiple immunosuppressant molecules and one or more multiple polymer conjugated with Siglec ligands can be conjugated together and then conjugated to one antigen molecule. Other molecule that can promote TB reg expansion (e.g. IL-2 and/or TGF-β and/or PD-L1) can also be conjugated.

Examples of sialic acid rich polymer-antigen conjugate for systemic lupus erythematosus are shown in FIG. 9. The sialic acid polymer-Antigen conjugate for SLE treatment has the structure of DNA-linker-sialic acid polymer. In one example, the patient having SLE will receive 200 mg˜1 g of the said conjugate as weekly i.v. injection to treat SLE.

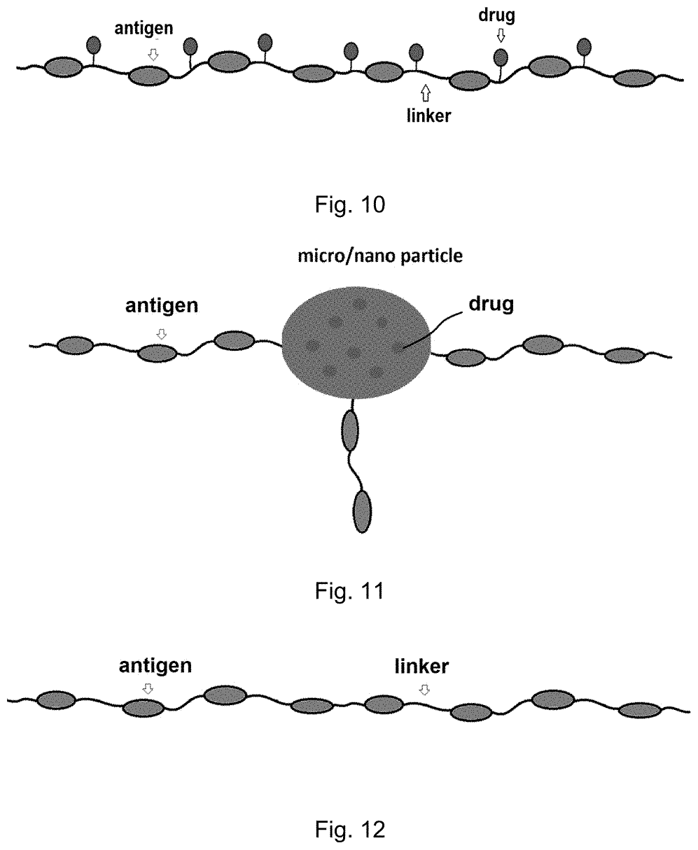

Another format is to connect multiple antigen/epitope with linkers to form a linear polymer and the drug (such as sialic acid or other immunosuppressant listed in the current invention including PD-L1) is conjugated to the linker region or antigen/epitope region or both as shown in FIG. 10. The linker can be either a synthetic polymer such as a PEG (e.g. MW 500D˜5KD) or a flexible peptide linker consist of hydrophilic amino acid such as -GGEGGGEGEEEGGGEGGEGGEEGGGEEDGG- (SEQ ID NO: 3). Example of suitable linker can be found in U.S. patent application Ser. Nos. 15/373,483; 15/169,640 and 62/517,994 by the current inventor. XTEN polypeptide from Amunix Inc. can also be used as a peptide linker. When peptide linker is used, the linear polymer can be expressed by recombinant technology if the antigen/epitope is also a peptide or protein that can be linked at its N and C terminal with linker. The drug can be conjugated to the linear polymer directly or with a second linker. The drug conjugated can be either as a single molecule form or multiple molecules form such as in a carrier or encapsulated in nano/micro particle form or in liposome form. In some embodiments, one or more PD-L1 is fused or conjugated with multiple antigen and linkers to form a fusion protein, which can be constructed by expression. Inhibitory ligand that can bind with inhibitory checkpoint receptor (e.g. A2AR, BTLA, CTLA-4, KIR, LAG3, TIM-3, VISTA, CD47 and etc) such as B7-H3, B7-H4 can also be used instead of PD-L1. In some embodiments, the number of antigen/epitope in each polymer backbone is more than 6, preferably more than 8. In some embodiments, the number of antigen/epitope conjugated to each polymer backbone is more than 10. In some embodiments, the number of drug conjugated to each polymer backbone is more than 4. In some embodiments, the number of drug conjugated to each polymer backbone is more than 8. The antigen can be either B cell antigen or T cell antigen in MHC-peptide complex form or the antigen peptide (or its derivative) that can bind with MHC or their combination.

Alternatively, one or more antigen/epitope containing polymer, which each contains one or more antigen/epitope, can be conjugated or coated to a nano/micro particle, which is encapsulated with immune suppressant drug and optionally antigen/epitope. Exemplary scheme can be seen in FIG. 11.

In some embodiments, the drug is not necessary. One format is to connect multiple antigen/epitope with linkers to form a linear polymer. The linker can be either a synthetic polymer such as a PEG (e.g. MW 500D˜5KD) or a flexible peptide linker consist of hydrophilic amino acid such as -GGEGGGEGEEEGGGEGGEGGEEGGGEEDGG- (SEQ ID NO: 3). Example of suitable linker can be found in U.S. patent application Ser. Nos. 15/373,483; 15/169,640 and 62/517,994 by the current inventor. XTEN polypeptide from Amunix Inc. can also be used as a peptide linker. When peptide linker is used, the linear polymer can be expressed by recombinant technology if the antigen/epitope is also a peptide or protein that can be linked at its N and C terminal with linker. Exemplary scheme can be seen in FIG. 12.

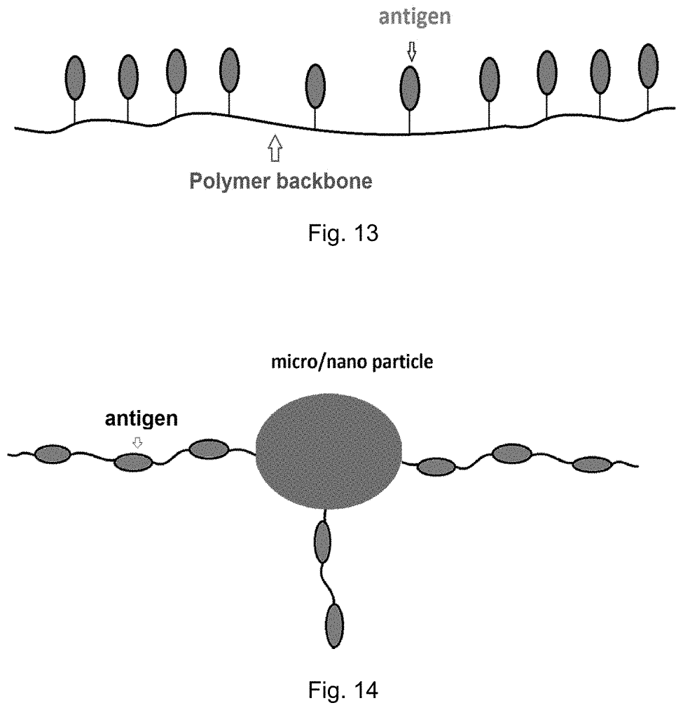

Another format is shown in FIG. 13, which is essentially multiple antigen/epitope conjugated to a polymer back bone (polymer carrier). The polymer back bone can be polypeptide such as Xten from Amunix, synthetic polymer such as poly acrylic acid, carbohydrate includes sialic acid containing polymer, hyaluronic acid, chondroitin sulfate, dextran, carboxyl dextran, cellulose, carboxyl cellulose and their derivatives. The polymer backbone used in previous described prodrug or in previous drug/antigen conjugate can be readily adopted. For example, the average MW of the carbohydrate or other polymer carrier is between 5KD˜1000KD. In some embodiments, the number of antigen/epitope conjugated to each polymer backbone is more than 8, preferably more than 10. The antigen/epitope can be conjugated to the polymer directly or via a linker. The linker can be either covalent or none-covalent. For example, the linker can be avidin conjugated on polymer bind with the biotin conjugated with antigen/epitope. In some embodiments, the polymer carrier is soluble in aqueous solution.

Similarly, one or more antigen/epitope containing polymer, which each contains one or more antigen/epitope, can be conjugated or coated to a nano/micro particle, which is optionally encapsulated with antigen/epitope. Exemplary scheme can be seen in FIG. 14.

The antigen can be either B cell antigen/epitope or T cell antigen/epitope (e.g. MHC-antigen peptide complex or conjugate; or the peptide antigen that can bind with MHC) or their combination. Examples of them can be found in the current application and related publications and patent applications.

Parvus' NAVACIM® technology use peptide-MHC coated nanoparticles (pMHC-NPs) to delete the high avidity cytotoxic effector T cells, expand a population of autoregulatory memory T cells to target and kill antigen presenting cells (APCs), expand and/or develop populations of Tr1 cells and/or B-regulatory cells in subject to treat corresponding autoimmune diseases. It is disclosed in publications and patent applications such as doi: 10.1016/j.immuni.2010.03.015; doi:10.1038/nature16962, doi: 10.1038/nnano.2017.56; doi: 10.1007/s00109-011-0757-z.; US patent application serial numbers U.S. Ser. No. 12/044,435, US20090155292A1, US20150125536A1, US20170333540A1, US20170095544A1 and US patent U.S. Pat. No. 8,354,110B2. It has been shown that mono specific pMHC-NP can expand cognate autoregulatory T cells or B cells, suppress the recruitment of noncognate specificities, prevent or treat autoimmunity disease.

The antigen/epitope (peptide-MHC complex such as NRP-V7-K^{d} or IGRP₂₀₆₋₂₁₄-K^{d} or both) used in these pMHC-NPs can also be used as antigen/epitope for the current invention to treat corresponding autoimmunity disease such as type 1 diabetes (T1D). Other T1D-relevant pMHC (peptide-MHC complex) can also be used as antigen/epitope for the current invention to treat type 1 diabetes (T1D). The pMHC (peptide-MHC complex) can be either autoimmune-disease-relevant peptides bound to major histocompatibility complex class II (pMHCII) molecule or autoimmune-disease-relevant peptides bound to major histocompatibility complex class I (pMHCI) molecule or their combinations. Examples of these peptide-MHC complex can be found in the prior arts listed above and can be readily used in the current invention to induce corresponding immune tolerance and to prevent/treat corresponding autoimmune disorder listed in the above cited prior arts.

The above cited prior arts use peptide-MHC-coated nanoparticles with diameter less than 100 nm. Bigger particles including micro particle can also be used to coat with peptide-MHC for the same application, e.g. 200 nm˜200 μm in diameter, as long as its surface are conjugated with high density of peptide-MHC complex, to generate pMHC-MPs (peptide-MHC-coated microparticles). In some preferred embodiments, it has a size of 500 nm˜10 μm in diameter with >0.5 peptide-MHC molecule/100 nm² surface area. Suitable particles can be made of biodegradable material such as PLGA. Example of biodegradable micro particle suitable for medical application and their surface conjugation protocol are well known to a skilled in the art and can be found easily in the publications.

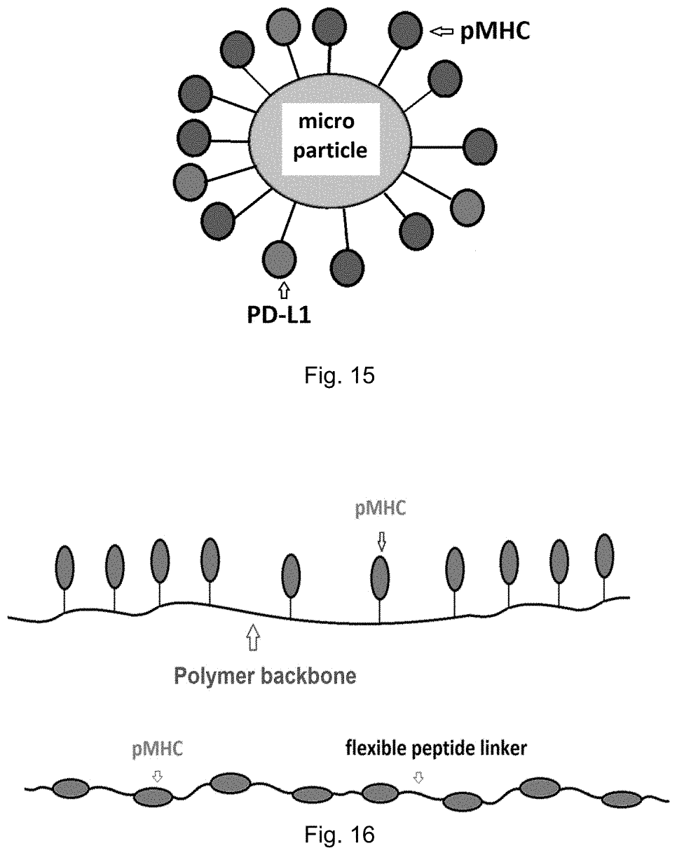

In some embodiments of the current invention and previous U.S. application Ser. Nos. 15/723,173, 16/380,951 and 16/029,594 by the current inventors, effector molecule such as immunosuppressant drug (e.g. rapamycin or PD-L1) can be further conjugated to or encapsulated in the pMHC coated nano/micro particle such as peptide-MHC-coated nanoparticles (pMHC-NPs) cited in the above prior arts (e.g. those used in Parvus' NAVACIM® technology) and those disclosed in the current invention to increase its efficacy. For example, the surface of pMHC-NPs or pMHC-MPs (peptide-MHC-coated microparticles) can be coated with PD-L1 (or its PD-1 binding domain or other PD-1 agonist). Conjugating PD-L1 can effectively inhibit cytotoxic T/B cell and boost Treg/Breg expansion. As shown in FIG. 15 coating additional T/B regulatory cell stimulating molecule/cytokine (e.g. PD-L1, IL-2, TGF-β et.ac.) to pMHC-NP or pMHC-MP is used to increase these T/B regulatory cell expansion and inactivate cytotoxic T/B cell directly. In another example, PD-L2 or other ligand for inhibitory immune check point receptor is coated to the surface of pMHC-NP or pMHC-MP. In another example, immunosuppressant drug such as rapamycin is conjugated to pMHC-NP/pMHC-MP or encapsulated within pMHC-NP/pMHC-MP. In one example, avidin coated NP or MP is prepared according to the protocol in Diabetes 2004 June; 53(6): 1459-1466. doi.org/10.2337/diabetes.53.6.1459. Next the mixture solution of biotinylated NRP-V7/H−2K^{d} and biotinylated PD-L1 is added to the avidin coated NP/MP in excess of the binding capacity of the coated avidin (e.g. 2˜5 folds excess) and incubated overnight at 4° C. Next the resulting pMHC-NP/pMHC-MP is washed with PBS 3 times to remove unbound protein. Bigger size NP (e.g. 100˜500 nm) coated with more avidin can also be used instead. Exemplary ratio of V7/H−2K^{d} vs biotinylated PD-L1 used can be between 10:1˜1:3. Other molecule that can promote T/B reg expansion (e.g. T/B reg promoting cytokines such as IL-2 and TGF-β) can also be co-coated to the NP or MP, e.g. by using biotinylated IL-2/TGF-β containing protein mixture described above. Other MHC-peptide complex such as IGRP₂₀₆₋₂₁₄-K^{d} can also be used instead to treat T1D. Other disease related MHC-peptide complex can also be used to treat corresponding disease, for example, pMOG₃₈₋₄₉/IA^{b} (disclosed in doi:10.1038/nature16962) coated NP or MP can also be encapsulated or coated with immunosuppressant to treat experimental autoimmune encephalomyelitis (EAE).

In some embodiments of the current invention, peptide-MHC-coated micro or nanoparticles (pMHC-NP/pMHC-MP) is prepared by coating recombinant single chain MHC complex on the surface of the NP/MP to treat the corresponding autoimmune diseases instead of the peptide-MHC complex described above. U.S. Ser. No. 08/596,387 disclosed single chain MHC complexes and uses thereof. U.S. Pat. No. 5,869,270 disclosed single chain MHC class II peptide fusion complexes with a presenting peptide covalently linked to the peptide binding grove of the complex. Eur J Immunol. 2000 December; 30(12):3522-32. disclosed recombinant human single-chain MHC-peptide complexes made from E. coli. A skilled in the art can readily adopt the peptide-recombinant single chain MHC complex/conjugate in the prior arts to prepare the peptide-recombinant single chain MHC complex/conjugate coated NP for the current invention. The term MHC complex includes both none-covalent MHC-peptide complex and covalent MHC-peptide conjugate such as those described above. Furthermore, mimetic or derivative of MHC-peptide complex can also be used in the current invention to replace the MHC-peptide complex as long as it can bind with the corresponding antigen specific TCR receptor. The MHC-peptide complex mimetic can be readily developed with phage display library or other screening method or computational modeling.

Another format is to use polymer-based peptide-MHC oligomer/multimer instead of peptide-MHC coated micro/nanoparticle to induce immune tolerance to the antigen of the MHC-peptide complex and to treat the corresponding autoimmune diseases. Preferably the MHC-peptide complex in each polymer is more than 6 copies. In some embodiments the MHC-peptide complex in each polymer is more than 8 copies. In some embodiments the MHC-peptide complex in each polymer is more than 20 copies. The polymer backbone can be a soluble polymer such as the polymer carrier and polymer backbone described in previous U.S. application Ser. Nos. 15/723,173, 16/380,951 and 16/029,594 by the current inventor. The polymer can be branched or linear. The polymer backbone can be polypeptide such as Xten from Amunix Inc, synthetic polymer such as poly acrylic acid, carbohydrate includes sialic acid containing polymer, hyaluronic acid, chondroitin sulfate, dextran, carboxyl dextran, cellulose, carboxyl cellulose and their derivatives. The polymer backbone used in previous described drug/antigen conjugate can be readily adopted. For example, the average MW of the carbohydrate or other polymer is between 5KD˜1000KD. The soluble polymer can be a linear polymer. Examples of those MHC multimer can be MHC pentamer, MHC dextramer (e.g. those from immudex.com) and those described in US 20100168390 A1: MHC multimers, methods for their generation, labeling and use. The administration protocol can be the same as the pMHC-NPs described above. For example, Immudex dextramer Cat no. WB3329 (peptide: VLFGLGFAI (SEQ ID NO:4); antigen: IGRP allele: HLA-A*0201) can be used to treat diabetes. In another example, Immudex unlabeled SA-Dextramer Cat No. DXO1 is used to mix with biotinylated NRP-V7/H−2K^{d} or the mixture of biotinylated NRP-V7/H−2K^{d} and biotinylated PD-L1 in excess (e.g. 1.2˜2 folds excess of the binding capacity of the streptavidin) and incubated overnight at 4° C. Next the resulting peptide-MHC polymer is dialyzed in PBS to remove unbound peptide-MHC. Other molecule that can promote TB reg expansion (e.g. IL-2 and/or TGF-β) can also be added to bind with SA-Dextramer, e.g. by using biotinylated IL-2/TGF-β containing protein mixture described above. The resulting pMHC multimer can be injected to a subject in need to treat diabetes T1D. Other MHC-peptide complex such as IGRP₂₀₆₋₂₁₄-K^{d} can also be used instead to build the pMHC multimer to treat T1D. The peptide-recombinant single chain MHC complex/conjugate and MHC-peptide complex mimetic can also be used as T cell antigen to build this kind of polymer for the same application. Multiple pMHC can be connected with a linker to build pMHC multimer. The linker can be either a synthetic polymer such as a PEG (e.g. MW 500D˜5KD) or a flexible peptide linker consist of hydrophilic amino acid. Example of suitable linker can be found in U.S. patent application Ser. Nos. 15/373,483; 15/169,640 and 62/517,994 by the current inventor. XTEN polypeptide from Amunix Inc. can also be used as a peptide linker. When peptide linker is used, the linear polymer can be expressed by recombinant technology if the antigen/epitope is also a peptide or protein that can be linked at its N and C terminal with linker. FIG. 16 shows an example of the scheme of multiple pMHC is conjugated or expressed in a polymer instead of being coated on particles.

The above MHC-peptide coated nanoparticle and dextramer based MHC-peptide complex use streptavidin/avidin to conjugate the MHC-peptide complex. Direct conjugation without streptavidin/avidin-biotin binding can also be used instead to incorporate the MHC-peptide complex to the NP/MP or linear polymer using chemical conjugation or other affinity binding such as Fc-protein A interaction. The site-specific conjugation is well known to the skilled in the art and can be adopted from related publications readily. For example, the surface micro/nanoparticle (MP/NP) or polymer can be modified/derivatized to have maleimide groups to allow the —SH (cysteine) of the peptide-MHC to conjugate to them using the well-known maleimide thiol reaction. The protocol for these kinds of modification, derivatization and conjugation are well known to the skilled in the arts and can be readily found in the publications and manual of the related reagents.

FIG. 16 shows the multiple pMHC is conjugated or expressed in a polymer instead of being coated on particles.

In some embodiments, the pMHC in either nano particle or micro particle or linear polymer form described above in either by current invention or prior arts can be used as a mixture with the immunosuppressive agent described above to treat corresponding pMHC specific autoimmune disease and allergy. The resulting composition to treat autoimmune disease and allergy contains therapeutically effective amount of pMHC and immunosuppressive agent physically mixed together. It can be injected as subcutaneous or intravenous or intralymphatic injection to treat the related dysfunction in a subject in need. The mixture can be in the form of powder, solution including high viscosity liquid or thermal phase changing formulation similar to those previously disclosed to achieve sustained release or implant. For example, a composition which is mixture of 0.1 mg˜5 mg rapamycin (either as nonencapsulated or encapsulated in nano or microparticle as those previously described) and 5˜25 mg above NRP-V7/H−2K^{d} containing linear polymer or nano or micro particle, can be injected to a subject in need once biweekly 3 times as subcutaneous injection or intramuscular injection or intravenous injection or intralymphatic injection to treat to treat diabetes. Viscosity enhancing agent such as 5% hyaluronic acid can also be added to the composition to achieve sustained release for subcutaneous injection or intramuscular injection or intralymphatic injection.

Human MHC class I and II are also called human leukocyte antigen (HLA). The most studied HLA genes are the nine classical MHC genes: HLA-A, HLA-C, HLA-DPA1, HLA-DPB1, HLA-DQA1, HLA-DQB1, HLA-DRA, and HLA-DRB1. In humans, the MHC gene cluster is divided into three regions: classes I, II, and III. The A, B and C genes belong to MHC class I, whereas the six D genes belong to class II. MHC alleles are expressed in codominant fashion. This means the alleles (variants) inherited from both parents are expressed equally. Each person carries 2 alleles of each of the 3 class-I genes, (HLA-A, HLA-B and HLA-C), and so can express six different types of MHC-I. In the class-II locus, each person inherits a pair of HLA-DP genes (DPA1 and DPB1, which encode α and β chains), a couple of genes HLA-DQ (DQA1 and DQB1, for α and β chains), one gene HLA-DRα (DRA1), and one or more genes HLA-DRβ (DRB1 and DRB3, -4 or -5). That means that one heterozygous individual can inherit six or eight functioning class-II alleles, three or more from each parent. There also non-classical MHC in human. Peptide MHC complex (pMHC) suitable for the current invention can be found from prior arts and publications readily. The peptide and MHC in the peptide MHC complex can be either covalently conjugated (or expressed) together or bound together to form a non-covalent complex. There are many autoimmune diseases related peptide MHC complex in human or animal being identified. For example, patent application serial numbers US20170095544A1, US20180127481A1, US20090155292A1 and US20150125536A1 disclosed disease specific peptide MHC complex, which can be really adopted for the current application. The MHC class I component can comprise all or part of a HLA-A, HLA-C, HLA-E, HLA-F, HLA-G molecule, particularly all or part of a HLA-A molecule, such as a HLA-A*0201 MHC class I molecule. The non-classical MHC class I component can comprise CD1-like molecules. An MHC class II component may comprise all or part of a HLA-DR, HLA-DQ, or HLA-DP. In certain aspects to treat autoimmune disease and allergy, the antigen/MHC complex is covalently or non-covalently coupled or attached to a substrate (antigen/MHC/particle complex or antigen/MHC/linear polymer). As used herein and unless specifically noted, the term MHC in the context of an pMHC complex intends a classical or a non-classical MHC class I protein and/or or classical or non-classical MHC class II protein, any loci of HLA DR, HLA DQ, HLA DP, HLA-A, HLA-B, HLA-C, HLA-E, CD1d, or a fragment or biological equivalent thereof, dual or single chain constructs, dimers (Fc fusions). In certain embodiments, the MHC class 1 component may comprise, consist essentially of, or alternatively further consist thereof all or part of a HLA-A, HLA-B, HLA-C, HLA-E, HLA-F, HLA-G or CD-1 molecule. In embodiments wherein the MHC component is a MHC class II component, the MHC class II component may comprise, consist essentially of, or alternatively further consist thereof all or a part of a HLA-DR, HLA-DQ, or HLA-DP. In certain embodiments, the MHC may comprise HLA DRB1, HLA DRB3, HLA DRB4, HLA DRB5, HLA DQB1, HLA DQA1, IAg7, I-Ab, I-Ad, HLA-DQ, HLA-DP, HLA-A, HLA-B, HLA-C, HLA-E or CD1d. Non-classical MHC molecules are also contemplated for use in MHC complexes of the disclosure. In some embodiments, non-classical MHC molecules are non-polymorphic, conserved among species, and possess narrow, deep, hydrophobic ligand binding pockets. These binding pockets are capable of presenting glycolipids and phospholipids to natural killer T (NKT) cells. NKT cells represent a unique lymphocyte population that co-express NK cell markers and a semi-invariant T cell receptor (TCR). They are implicated in the regulation of immune responses associated with a broad range of diseases.

The T cell recognize T cell antigen by its TCR receptor. The T cell antigen normally is in the form of MHC-epitope binding complex. The epitope normally is a peptide (sometimes other molecules such as carbohydrate) processed by APC. In the current invention, the antigen for T cells can be the formed MHC-epitope complex or its fragment/derivatives/mimics, which has higher specific affinity to TCR than the epitope alone. It can be the monomer form or oligomer (dimer, trimer, tetramer, pentamer or even higher degree polymer) form such as the MHC tetramer currently used in research to label immune cells. For example, the tetramer (e.g. those in doi: 10.1073/pnas.0508621102) can be conjugated with the cell inactivating agent with an optional linker to treat Type 1 diabetes in human by inactivating the autoimmune T cell. The epitope (e.g. peptide) can be covalently conjugated with MHC to increase its stability by well-known means as disclosed in well-known publications. Similarly, the antigen used for B cell in the current invention can also be oligomer or polymer form. However, sometimes the antigen used for B cell inactivation do not require the MHC component.

The current invention also discloses an autologous immune cell therapy method to treat autoimmune disease, allergy, inhibit anti-drug antibody production or induce antigen specific immune tolerance in a subject. It comprises the following steps: autologous immune cell collection and separation from a subject in need, stimulating with disease related pMHC complex to expand antigen specific regulatory immune cell and/or inhibitory immune cells in vitro to reach a desired number of target cells, and then infuse back the expanded autologous immune cell to the subject for desired therapeutical effect. Optional antigen specific immune cell selection step and immune cell type selection step can also be performed. And optional immune cell expansion with stimulating molecule other than pMHC complex can also be performed together with pMHC complex or separately. The source of autologous immune cell collection and separation from a subject can be bone marrow or lymph node extract or blood or blood fraction from the said subject or their combinations. In some embodiments, one can separate the lymphocyte from the blood of the subject in need with blood cell separator and/or leukapheresis. For example, 200 ml blood is draw from the patient and the lymphocyte is collected by using a blood cell separator on this 200 ml blood. The procedure of lymphocyte collection from blood is well known to the skilled in the art. It can be performed using commercial blood cell separator. The resulting lymphocyte contains B cell and T cell and possibly other white blood cells. Optionally the B cell can be further removed, e.g. with a cell sorter such as FACS or magnetic particles coated with B cell surface marker specific antibody, there are many commercial kits and instruments available for this purpose and the procedure is well known to the skilled in the art. However, in other embodiments the B cells are desired to stay in order to convert them to Breg cells. In the current invention inhibitory immune cells that can inhibit immune function is considered as regulatory immune cell, therefore regulatory immune cell includes both antigen specific regulatory immune cell and non-antigen specific inhibitory immune cells.

Next the collected B/T cell containing lymphocyte is stimulated with disease (e.g. autoimmune disease or allergy or the antigen for immune tolerance inducing) related (specific) pMHC complex to expand the antigen specific regulatory immune cell and/or inhibitory immune cells within the collected lymphocyte in vitro by culturing with effective amount of pMHC complex. Suitable pMHC complex include the polymer based pMHC multimer or peptide-MHC coated micro/nanoparticle disclosed above and previous U.S. patent application Ser. Nos. 15/723,173, 16/380,951 and 16/029,594 by the current inventor or the peptide-MHC coated nanoparticle disclosed in the prior arts (e.g. those in doi: 10.1007/s00109-011-0757-z; doi: 10.1016/j.immuni.2010.03.015; doi:10.1038/nature16962; doi: 10.1038/nnano.2017.56; patent application serial numbers US20170095544A1, U.S. Ser. No. 12/044,435 and US20150125536 A1) and those used in Parvus' NAVACIM® technology.

Because for most autoimmune diseases sometimes multiple autoantigens are involved for one disease (e.g. GAD65, insulin, preproinsulin and sometimes other antigens for type 1 diabetics, and for each antigen sometimes multiple epitopes fragment for each antigen are the source causing diseases), therefore in some embodiments the pMHC complex to expand antigen specific regulatory immune cell and/or inhibitory immune cells in vitro for corresponding autoimmune disease can be a mixture of different pMHC complex carrying different autoantigen and for each antigen its different epitope for this disease, such as a mixture of different polymer based pMHC multimer or different peptide-MHC-coated micro/nanoparticle related to the target disease to be treated.

In some embodiments, the disease-relevant antigens are:

one or more diabetes-relevant antigens and is derived from an antigen selected from one or more of the group: preproinsulin (PPI), islet-specific glucose-6-phosphatase (IGRP), glutamate decarboxylase (GAD), islet cell autoantigen-2 (ICA2), insulin, proinsulin, or a fragment or an equivalent of each thereof, and their combinations;

one or more multiple sclerosis-relevant antigen and is derived from an antigen selected from one or more of the group: myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins, NOGO A, glycoprotein Po, peripheral myelin protein 22, 2′3′-cyclic nucleotide 3′-phosphodiesterase, or a fragment or an equivalent of each thereof, and their combinations;

one or more celiac disease-relevant antigen and is derived from gliadin or a fragment or an equivalent thereof, and their combinations, and their combinations;

one or more primary biliary cirrhosis-relevant antigen and is derived from PDC-E2 or a fragment or an equivalent thereof, and their combinations;

one or more pemphigus folliaceus-relevant antigen and/or pemphigus vulgaris-relevant antigen and is derived from an antigen selected from one or more of the groups: DG1, DG3, or a fragment or an equivalent of each thereof, and their combinations;

one or more neuromyelitis optica spectrum disorder-relevant antigen and is derived from AQP4 or a fragment or an equivalent thereof, and their combinations;

one or more arthritis-relevant antigen and is derived from an antigen selected from one or more of the group: heat shock proteins, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, Complement C3, or a fragment or an equivalent of each thereof, and their combinations;

one or more allergic asthma-relevant antigen and is derived from an antigen selected from one or more of the group: DERP1, DERP2, or a fragment or an equivalent of each thereof, and their combinations;

one or more inflammatory bowel disease-relevant antigen and is derived from an antigen selected from one or more of the groups: flagelin, Fla-2, Fla-X, YIDX, bacteroides integrase, or a fragment or an equivalent of each thereof, and their combinations;

one or more systemic lupus erythematosus-relevant antigen and is derived from an antigen selected from one or more of the group: double-stranded (ds)DNA, ribonucleoprotein (RNP), Smith (Sm), Sjögren's-syndrome-related antigen A (SS-A)/Ro, Sjögren's-syndrome-related antigen B (SS-B)/La, R060, R052, histones, or a fragment or an equivalent of each thereof, and their combinations;

one or more atherosclerosis-relevant antigen and is derived from an antigen selected from one or more of the groups: ApoB, ApoE or a fragment or an equivalent of each thereof, and their combinations;

one or more COPD-relevant antigen and/or emphysema-relevant antigen and is derived from elastin or a fragment or an equivalent thereof, and their combinations;

one or more psoriasis-relevant antigen and is derived from an antigen selected from one or more of the groups: Cap18, ADMTSL5, ATL5, or a fragment or an equivalent of each thereof, and their combinations;

one or more autoimmune hepatitis-relevant antigen and is derived from an antigen selected from one or more of the groups: CYP2D6, SLA, or a fragment or an equivalent of each thereof; and their combinations;

one or more Sjogren's Syndrome-relevant antigen and is derived from an antigen selected from one or more of the groups: (SS-A)/Ro, (SS-B)/La, MR3, R060, R052, or a fragment or an equivalent of each thereof; and their combinations;

one or more scleroderma-relevant antigen and is derived from an antigen selected from one or more of the groups: CENP-C, TOP 1, RNA polymerase III, or a fragment or an equivalent of each thereof, and their combinations;

one or more anti-phospholipid syndrome-relevant antigen and is derived from APOH or a fragment or an equivalent thereof, and their combinations;

one or more ANCA-associated vasculitis-relevant antigen and is derived from an antigen selected from one or more of the groups: MPO, PRTN3, or a fragment or an equivalent of each thereof, and their combinations;

one or more Stiff Man Syndrome-relevant antigen and is derived from GAD or a fragment or an equivalent thereof.

Examples of the sequence of these antigen peptides can be readily found in patent application serial numbers US20170095544A1, US20090155292A1 and other prior arts. For example, diabetes-relevant antigens include but are not limited to those derived from PPI, IGRP, GAD, islet cell autoantigen-2 (ICA2), and/or insulin. Autoreactive, diabetes-relevant antigenic peptides include, but are not limited to, include those listed in the following, in addition to the peptides and proteins disclosed in U.S. patent publication No. 2005/0202032, as well as equivalents and/or combinations of each thereof. For example, they can be the antigens disclosed in U.S. patent application serial number U.S. Ser. No. 10/124,045B2 as below:

GAD65114-123, GAD65536-545, GFAP143-151, GFAP214-222, IA-2172-180, IA-2482-490, IA-2805-813, ppIAPPs5i3, ppIAPP9-17, IGRP152-160, IGRP211-219, IGRP215-223, IGRP222-230, IGRP228-236, IGRP265-273, IGRP293-301, proinsulinL2-10, proinsulinL3-11, proinsulinL6-14, proinsulinB5-14, proinsulinB10-18, proinsulinB14-22, proinsulinB15-24, proinsulinB17-25, proinsulinB18-27, proinsulinB20-27, proinsulinB21-29, proinsulinB25-C1, proinsulinB27-05, proinsulinC20-28, proinsulinC25-33, proinsulinC29-A5, proinsulinA1-10, proinsulinA2-10, proinsulinA12-20, hInsB10-18, hIGRP228-236, hIGRP265-273, IGRP206-214, hIGRP206-214, NRP-A7, NRP-I4, NRP-V7, YAI/Db, INS B15-23, PPI76-90 (K88S), IGRP13-25, GAD555-567, GAD555-567(557I), IGRP23-35, B24-C36, PPI76-90, INS-I9, TUM, G6Pase, proinsulinL2-10, proinsulinL3-11, proinsulinL6-14, proinsulinB5-14, proinsulinB10-18, proinsulinB14-22, proinsulinB15-24, proinsulinB17-25, proinsulinB18-27, proinsulinB20-27, proinsulinB21-29, proinsulinB25-C1, proinsulinB27-05, proinsulinC20-28, proinsulinC25-33, proinsulinC29-A5, proinsulinA1-10, proinsulinA2-10, and proinsulinA12-20.

In certain aspects, the human disease and disease related pMHC complex used for the treatment can be:

type I diabetes and the pMHC complex is selected from the group of: insB10-18-HLA-A2, PPI76-90(K885)-HLA-DRB1*0401/DRA, IGRP13-25-HLA-DRB1*0301/DRA, GAD555-567-HLA-DRB1*0401/DRA, GAD555-567(557I)-HLA-DRB1*0401/DRA, IGRP23-35-HLA-DRB1*0401/DRA, B24-C36-HLA-DRB1*0301/DRA, or PPI76-90-HLA-DRB1*0401/DRA;

multiple sclerosis and the pMHC complex is selected from the group of: MBP86-98-HLA-DRB1*1501/DRA, MBP89-101-HLA-DRB5*0101/DRA, MOG38-52-HLA-DRB4*0101/DRA, MOG97-109(E1075)-HLA-DRB1*0401/DRA, MOG203-217-HLA-DRB3*0101/DRA, PLP54-68-HLA-DRB3*0101/DRA, PLP94-108-HLA-DRB1*0301/DRA, PLP250-264-HLA-DRB4*0101/DRA, MPB13-32-HLA-DRB5*0101/DRA, MPB83-99-HLA-DRB5*0101/DRA, MPB111-129-HLA-DRB5*0101/DRA, MPB146-170-HLA-DRB5*0101/DRA, MOG223-237-HLA-DRB3*0202/DRA, MOG6-20-HLA-DRB5*0101/DRA, PLP88-102-HLA-DRB3*0202/DRA, or PLP139-154-HLA-DRB5*0101/DRA;

Celiac disease and the pMHC complex is selected from the group of: aGlia57-68-HLA-DQA1*0501/HLA-DQB1*0201, aGlia62-72-HLA-DQA1*0501/HLA-DQB1*0201, aGlia217-229-HLA-DQA1*0501/HLA-DQB1*0302, or aGlia217-229-HLA-DQA1*03/HLA-DQB1*0302;

primary biliary cirrhosis and the pMHC complex is selected from the group of: PDC-E2122-135-HLA-DRB4*0101/DRA, PDC-E2249-262-HLA-DRB4*0101/DRA, PDC-E2249-263-HLA-DRB1*0801/DRA, PDC-E2629-643-HLA-DRB1*0801/DRA, PDC-E272-86-HLA-DRB3*0202/DRA, PDC-E2353-367-HLA-DRB3*0202/DRA, PDC-E2422-436-HLA-DRB3*0202/DRA, PDC-E2629-643-HLA-DRB4*0101/DRA, PDC-E280-94-HLA-DRB5*0101/DRA, PDC-E2353-367-HLA-DRB5*0101/DRA, or PDC-E2535-549-HILA-DRB5*0101/DRA, mPDC-E2166-181-I-Ag7, or mPDC-E282-96-I-Ag7;

neuromyelitis optica spectrum disorder and the pMHC complex is selected from the group of: AQP4284-298-HLA-DRB1*0301/DRA, AQP463-76-HLA-DRB1*0301/DRA, AQP4129-143-HLA-DRB1*0401/DRA, or AQP439-53-HLA-DRB1*1501/DRA;

allergic asthma and the pMHC complex is selected from the group of: DERP-116-30-HLA-DRB1*0101/DRA, DERP-116-30-HLA-DRB1*1501/DRA, DERP1171-185-HLA-DRB 1*1501/DRA, DERP-1110-124-HLA-DPB1*0401/DRA, DERP-226-40-HLA-DRB1*0101/DRA; DERP-226-40-HLA-DRB1*1501/DRA, or DERP-2107-121-HLA-DRB1*0301/DRA;

The MHC used to build pMHC can be either MHC class I or MHC class II, preferably includes both MHC class I and MHC class II, therefore be able to expand both CD4+ and CD8+ Treg in vitro. In the mixture of pMHC complex used to expand regulatory immune cells each species of polymer based pMHC multimer or peptide-MHC-coated micro/nanoparticle will carry multiple copies of either peptide-MHC I complex or peptide-MHC II complex. Within each species, the peptide can be the same or different but need to be related to the same diseases and it need to be able to bind with the corresponding MHC I or MHC II. In some embodiments within each species, the MHC is also the same or the same class (either MHC I or MHC II). In different species the peptides are different. In the current invention, both polymer based pMHC multimer and peptide-MHC coated micro/nanoparticle are essentially pMHC multimer containing multiple copies of pMHC despite they are in either polymer form or particle form. In the following descriptions, both polymer based pMHC multimer and peptide-MHC coated micro/nanoparticle are called pMHC multimer and the term pMHC multimer include both polymer based pMHC multimer and peptide-MHC coated micro/nanoparticle. For example, for NOD mice expressing MHC I K^{d} and MHC II IA⁷, the pMHC multimer to be used can be a mixture selected from NRP-V7/K^{d} pMHC multimer, IGRP₂₀₆₋₂₁₄/K^{d} pMHC multimer, 2.5 mi/IA^{g7} pMHC multimer, IGRP₄₋₂₂/IA^{g7} pMHC multimer and IGRP₁₂₈₋₁₄₅/IA^{g7} pMHC multimer. In some embodiments preferably, the number of copies of pMHC in each pMHC multimer is >6. In some embodiments preferably, the copy number of pMHC in each pMHC multimer is >8. In some embodiments preferably, the copy number of pMHC in each pMHC multimer is >10. In some embodiments preferably, the copy number of pMHC in each pMHC multimer is >20. In some preferred embodiments, the pMHC particle has a surface pMHC density >0.5 copies of pMHC complex/100 nm² surface area. In some preferred embodiments, the pMHC particle has a surface pMHC density >2 copies of pMHC/100 nm² surface area. The concentration of pMHC multimer used to expand regulatory immune cells in vitro can be between 0.1 ug/ml to 10 mg/ml in the culture media. In one example the collected immune cells from the subject is cultured in complete medium for pMHC multimer specific cell expansion, which consisted of 10% heat-inactivated fetal bovine serum (Biosource International), nonessential amino acids, 0.5 mM sodium pyruvate, 5 mM Hepes, 1 mM glutaMax I (all from Invitrogen), one or more species of pMHC multimer for a specific disease each at 50 ug/ml in DMEM base. The culture is monitored daily and maintained at 0.7×10⁶˜1×10⁶/ml by diluting with complete medium for 8˜12 days or until desired amount of target cells are obtained. Optionally 200˜2000 IU/mL IL-2/anti-IL-2 mAb (e.g. those described in DOI: 10.4049/jimmunol.1402540) can be included into the medium. Optionally previously described immunosuppressant molecule that can promote T/B reg expansion and/or inhibit harmful auto reactive T/B cell (e.g. rapamycin, methotrexate, retinoic acid, TGF-β, agonist for CTLA-4, 4-1BB ligand such as those in doi.org/10.4049/jimmunol.179.11.7295, agonist for PD-1 such as PD-L1, PD-L2, IL-15, IFN-y, IL-10, IL-21, IL-27, IL-4, IL-2/anti-IL-2 antibody complexes or their mimics or derivatives such as a pegylated IL-2 NKTR-358) can also be added to the culture medium. For example, 10 ng/mL˜1 ug/mL rapamycin can be included in the culture medium.

In some embodiments anti-CD3 antibody coated beads and/or anti-CD3 antibody coated beads are added to the culture medium without the addition of anti-CD28 antibody coated beads or anti-CD28 antibody coated beads. In some embodiments anti-CD28 antibody coated beads and/or anti-CD28 antibody coated beads are added to the culture medium without the addition of anti-CD3 antibody coated beads or anti-CD3 antibody coated beads. Anti-CD3 antibody, anti-CD3 antibody coated beads, anti-CD3 antibody and anti-CD3 antibody coated beads are commercially available and widely used culturing immune cells. The protocol is well-known to the skilled in the art.

Optionally at the later stage of culturing, anti-CD3 antibody coated beads and anti-CD28 antibody coated beads can also be added to further stimulate cell expansion. Artificial antigen presenting cells (aAPCs) such as those described in the prior arts including doi: 10.1126/scitranslmed.3001809 can also be used for cell expansion.

Optionally after the cell expansion step an expression of FoxP3 step can be performed such as using retrovirus-mediated expression of FoxP3 to the expanded regulatory cells such as regulatory T cells, example of the expression protocol of lentivirus-mediated expression of FOXP3 can be found at doi.org/10.4049/jimmunol.175.5.3053. As FOXP3 is not a surface marker, a non-T cell surface marker can be introduced to the retrovirus-mediated expression system under the control of FoxP3 promotor, therefore once the FoxP3-transduced T-cells express FoxP3, it will express the non-T cell surface marker to allow the sorting/isolation of FoxP3+ cells based on this non-T cell surface marker. Preferably the non-T cell surface marker should not inhibit the regulatory activity of T cells and is an endogenous protein of the target species, which will not cause rejection of the target host after adoptive transfer; for example, CD19 or CD 20 or CD22 or CD34 or CD235a can be used as surface marker for FOXP3 expression.

An optional sorting step on the basis of differential expression of surface markers of regulatory immune cells can also be performed to isolate the desired regulatory immune cells and to remove the unwanted effector immune cells either before or after the above cell expansion step or both, for example, the antigen binding^{(+ with low avidity)} CD8⁺CD25⁻CD44^{hi}CD122⁺ cells (as those described in doi:10.1016/j.immuni.2010.03.015), antigen binding ⁺CD4⁺CD25⁻Tr1 cells (as those described in doi:10.1038/nature16962 and doi:10.1038/NNANO.2017.56) and antigen binding ⁺CD4⁺CD25⁺CD127^{(low/-)} Treg cells can be isolated for adoptive transfer using well known method such as flowcytometry with fluorescent dye labeled antibody against cell surface marker (FACS) and/or magnetic particles coated with antibody against cell surface maker; the antigen specific cell can be selected using fluorescent dye labeled pMHC tetramer or polymer based pMHC multimer followed by FACS; or pMHC coated magnetic particle followed by magnetic separation; and preferably using the mixture of different pMHC multimer carrying different antigen peptide of the same disease to isolate a mixture containing different cell clones targeting the same disease but different epitope or different antigen. The selection can be the combination of positive and negative selection. In some embodiments, a faction of antigen binding ^{(+ with low avidity)}CD8⁺CD25⁻CD44^{hi}CD122⁺ cells are further selected based on their additional surface marker property of CD69⁻ or CCR7⁻ or CXCR3⁺ or CD62L^{hi} or their combinations for later adoptive transfer. In some embodiments, a faction of antigen binding ⁺CD4⁺CD25⁻Tr1 cells are further selected based on their additional surface marker property of CD49b⁺ or LAG-3⁺ or CD44^{hi} or CD62L^{low} or their combinations for later adoptive transfer.

The sorting/isolation of cells based on their surface markers is well known to the skilled in the art and there are many known kits, reagent and protocols can be adopted for the current application. For example, CD4⁺CD25⁺CD127^{(low/-)} Tregs can be isolated on a BD FACSAria II high-speed cell sorter housed in a class 10,000 clean room with the following GMP-grade lyophilized antibodies: CD4-PerCP (peridinin chlorophyll protein) (L200), CD127-PE (phycoerythrin) (40131), and CD25-APC (allophycocyanin) (2A3) (BD Biosciences). The sorted CD4⁺CD25⁺CD127^{(low/-)} T cells are collected into 3 ml of X-VIVO 15 medium (Lonza, catalog no. 04-418Q) containing 10% human heat-inactivated pooled AB serum (Valley Biomedical). Treg populations are analyzed for purity after sort and determined based on the percentage of CD4⁺CD127^{(low/-)} CD25⁺ T cells. The selection of antigen (e.g. using pMHC multimer) specific T cells can also be performed. The selection of antigen specific T cells can be done at the same time of selecting CD4⁺CD25⁺CD127^{(low/-)} cells using mixture of different pMHC multimer labeled with same fluorescence dye compatible with BD FACSAria and orthogonal to the fluorescent tag for CD4⁺CD25⁺CD127^{(low/-)} markers. The isolation of antigen specific cells and CD4⁺CD25⁺CD127^{(low/-)} cells can also be done sequentially, for example CD4⁺CD25⁺CD127^{(low/-)} cells are isolated first as described above, next a mixture of NRP-V7/K^{d} pMHC multimer, IGRP₂₀₆₋₂₁₄/K^{d} pMHC multimer, 2.5 mi/IA^{g7} pMHC multimer, IGRP_{4-22/}M^{g7} pMHC multimer and IGRP_{128-145/}IA^{g7} pMHC multimer, all labeled with phycoerythrin are used to selected the antigen binding+T cells from the above CD4⁺CD25⁺CD127^{(low/-)} cells. An optional cell expansion step by in vitro culturing can also be performed after selection. This will generate antigen binding+CD4⁺CD25⁺CD127^{(low/-)} T reg to treat T1D in NOD mice. The pMHC multimer can be pMHC tetramer, pentamer, dextramer, polymer with higher degree of pMHC or pMHC coated particle described above.

In the last step, effective amount of adoptive transfer of the resulting regulatory immune cells (e.g. >1×10⁶ cells or >1×10⁸ cells) back to the subject in need is performed. The adoptive transferred cells will induce antigen specific tolerance in vivo, therefore can be used to treat corresponding autoantigen specific autoimmune disease or allergy. The adoptive transfer can be performed periodically such as once every 3 months or once per 6 months or be performed as needed. In some embodiments, preferably >1×10⁷ copied of cells are adoptive transferred to patient in need. In some embodiments, preferably >1×10⁸ copied of cells are adoptive transferred to patient in need. In some embodiments, preferably >1×10⁹ copied of cells are adoptive transferred to patient in need.

The method described above uses said prepared autologous regulatory immune cell using disease related pMHC to adoptive transfer to the donor to treat said pMHC related disease. Alternatively, using the same procedure the said prepared regulatory immune cell can also be adoptive transferred to a second person to treat said pMHC related disease the second person has. Therefore, the current method also disclosed an allogeneic immune cell therapy method to treat autoimmune disease, allergy, inhibit anti-drug antibody production or induce antigen specific immune tolerance in a subject. It comprises the following steps: immune cell collection and separation from a first subject as donor, stimulating with pMHC complex to expand antigen specific regulatory immune cell and/or inhibitory immune cells in vitro to reach a desired number of target cells, and then infuse back the expanded allogeneic regulatory immune cell to a second subject for desired therapeutical effect. The donor can be either a healthy donor or a subject having said disease. These allogeneic regulatory immune cells can be engineered to express PD-L1, which will enhance their immune inhibiting activity. The method and protocol of PD-L1 genetic overexpression can be adopted from well-known prior arts and publications such as those described in DOI: 10.1126/scitranslmed.aam7543. The allogeneic regulatory immune cells can also be engineered to delete their PD-1 expression to avoid the interference from their expressed PD-L1. Other immune suppression cytokine can also be over expressed by genetic modification to these allogeneic regulatory immune cells, such as IFN-gama, IL-10, IL-21, IL-35 and TGF beta, and optionally their endogenous receptors for these immune suppression cytokines can be deleted by genetic editing. Optionally introducing FOXP3 expression with retro virus can also be performed. They can be engineered to not to express MHC on their surface and also to express KIR inhibitory ligand such as HLA-E, HLA-G with optional human cytomegalovirus (HCMV) glycoprotein UL40 or its fragment to reduce the host rejection. These genetic engineering can be performed either before expansion or after expansion and before adoptive transfer.

Similar to the strategy for SLE treatment described in the patent application serial number U.S. Ser. No. 15/883,100 by the current inventor, a companion test such as ELISA test can be performed to the patient to identify the antigens and MHC alleles involved in the disease and use this information to select suitable pMHC multimer by using the identified antigens and MHC alleles to construct pMHC.

The methods described above use the mixture of pMHC I and pMHCII multimer to stimulate/expand CD8+ cells and CD4+ cells at the same time. Alternatively, leukocytes collected from a donor subject (for allogeneic transfer) or a subject in need (for autologous transfer) are first divided to two groups: CD4+ group and CD8+ group by flowcytometry sorting or magnetic separation. Next the stimulation/expansion are performed separately. For CD4+ cells pMHC II multimers are used and for CD8+ cells pMHC I multimers are used. For example, a mixture of NRP-V7/K^{d} pMHC multimer, IGRP₂₀₆₋₂₁₄/K^{d} pMHC multimer is added to the CD8+ cell; a mixture of 2.5 mi/IA^{g7} pMHC multimer, IGRP_{4-22/}IA^{g7} pMHC multimer and IGRP_{128-145/}IA^{g7} pMHC multimer is added to CD4+ cell. PD-L1, PD-L2 (e.g. 0.1 ug/ml to 100 ug/mL) or their derivatives such as those described in doi: 10.1084/jem.20090847, patent applications US20160040127A1 and US20120076805A1 can be included in CD8+ cell cultural medium or CD4+ cell cultural medium or both to stimulate regulatory T cell expansion and to inhibit effect T cell activity. Optionally previously described immunosuppressant and molecule that can promote T/B reg expansion and/or inhibit harmful auto reactive T/B cell (e.g. rapamycin, methotrexate, retinoic acid, agonist for CTLA-4, 4-1BB ligand such as those in doi.org/10.4049/jimmunol.179.11.7295, TGF-β, IL-15, IFN-γ, IL-10, IL-21, IL-27, IL-4, IL-2/anti-IL-2 antibody complexes or their mimics or derivatives such as a pegylated IL-2 NKTR-358) can also be added to the culture medium. The target cell to be expanded in CD8 cell populations are antigen binding ^{(+ with low avidity)}CD8⁺CD25⁻CD44^{hi}CD122⁺ cells. The target cell to be expanded in CD4 cell populations are antigen binding ⁺CD4⁺CD25⁻Tr1 cells and antigen binding ⁺CD4⁺CD25⁺CD127^{(low/-)} Treg cells. Optional stimulation with anti-CD3, anti-CD28, 4-1BBL, 4-1BB agonist, artificial antigen presenting cells (aAPCs); using retrovirus-mediated expression of FoxP3; sorting step on the basis of differential expression of surface markers of regulatory immune cells can also be performed on the CD4+ cell fraction and CD8+ cell fraction separately as previously described for the CD4+CD8+ mixture. Next the desired amount of CD4+ and CD8+ target cells are obtained; they are combined and adoptive transferred back to the subject in need to treat diabetes. It can be either autologous transfer or allogeneic transfer. If allogeneic transfer is involved, genetic engineering described in paragraph 0084 can be performed.

In some embodiments, first the disease related antigen specific immune cells are isolated from leukocytes collected from a donor subject (for allogeneic transfer) or a subject in need (for autologous transfer) using a mixture of fluorescent dye labeled disease related pMHC multimers and FACS, or using magnetic particles coated with high density of pMHC with magnetic separation. Or leukocytes collected from a donor subject (for allogeneic transfer) or a subject in need (for autologous transfer) are stimulated with disease related pMHC multimers first to expand the disease related antigen specific cells and then the disease related antigen specific cells are isolated from said stimulated leukocytes using a mixture of fluorescent dye labeled disease related pMHC multimers and FACS, or using magnetic particles coated with high density of pMHC with magnetic separation. Preferably, the copy number of pMHC in each pMHC multimer is >8. The isolated cells include a mixture of regulatory T cells and effector T cells and helper T cells specific to autoantigens (e.g. HLA-A2insB10-18 for diabetic patient). Optionally this mixture of cells can be treated by in vitro conversion by introducing FOXP3 expression into the cells (e.g. those described above and that in Molecular Therapy (2007) 16, no.1, 194-202) to covert some of the effector cells into Treg. Next antigen specific regulatory T cells are isolated from this mixture of cells by either removing the effector T cells from the population or positively selecting the regulatory T cells or the combination. The effector T cells can be removed from the mixture of cells by their surface expressed makers with positive selection method using well know means such as magnetic beads/flow cytometry/affinity columns specific to effector T cells' surface markers. The T reg can be purified/isolated based on its surface maker with well-known method such as those described above and those in the publications and the commercial kits (e.g. those described in www.bdbiosciences.com/us/applications/research/t-cell-immunology/regulatory-t-cells/m/745680/workflow/tregenrichment). The isolated/purified Treg can be then in vitro expanded with standard T cell expansion reagents such as anti-CD3, anti-CD28, artificial antigen presenting cells (aAPCs) and IL-2 as previously described optionally with additional added pMHC multimer and optionally added immunosuppressant molecules. The expanded Treg can be optionally further purified again; optionally the Treg can also be treated by in vitro conversion by introducing FOXP3 expression into the cells (e.g. those described in Molecular Therapy (2007) 16, no.1, 194-202); and then injected back to the patient to treat related autoimmune disease. This resulting poly colonial T regs are antigen specific, therefore provide better efficacy and lower off target effect for the target disease treatment. This method increases Treg cells in the patient for disease specific antigens therefore provide a treatment effect for the autoimmune disease or inducing immune tolerance. The resulting mixture of Treg specific to multiple autoantigens involved in a specific disease can be prepared and injected to the patient having auto immunity to one or more of these autoantigens. The Treg can also inhibit the corresponding B cells to inhibit the autoantibody production if the T cell antigen is derived from that B cell antigen from APC. Therefore, the Treg can also be used to treat auto immunity generated by autoimmune B cell/autoantibody. The isolated leukocytes or disease related antigen specific cells or expanded disease related antigen specific cells can also be divided to two groups: CD4+ and CD8+ cells as previously described and then treated accordingly to isolate/expand CD4+ regulatory cells and CD8+ regulatory cells separately and then are combined for adoptive cell transfer. It can be either autologous transfer or allogeneic transfer. If allogeneic transfer is involved, genetic engineering described in paragraph 0084 can be performed.

In some embodiments, the leukocytes collected from a donor subject (for allogeneic transfer) or a subject in need (for autologous transfer) or these leukocytes that are further stimulated with disease related pMHC multimers to expand the disease related antigen specific cells; the antigen binding ^{(+ with low avidity)}CD8⁺CD25⁻CD44^{hi}CD122⁺ cells, antigen binding ⁺CD4⁺CD25⁻Tr1 cells and antigen binding ⁺CD4⁺CD25⁺CD127^{(low/-)} Treg cells can be isolated with FACS or magnetism based separation using the methods previously described, by selecting from either the mixture of CD4+ and CD8+ cells or divide them into CD4+ and CD8+ group and then perform the selection separately on these two sub sets. The Treg can also be isolated based on their cytokine secretion profile using the well-known method and protocol. These isolated antigen specific T cells including Treg are then expanded in vitro using well known protocol such as using anti-CD3, anti-CD28, 4-1BBL, 4-1BB agonist, artificial antigen presenting cells (aAPCs) and IL-2 as previously described with optionally added pMHC multimer and optionally added previously described immunosuppressant molecules. The resulting expanded regulatory cells are then adoptively transferred back to the target in need to treat the related disease. It can be either autologous transfer or allogeneic transfer. If allogeneic transfer is involved, genetic engineering described in paragraph 0084 can be performed. For example, to further expand the target cells, the isolated antigen specific Tregs are cultured with either GMP anti-CD3/CD28 mAb-coated Dynabeads (3:1 bead:cell) or with K562 cell lines engineered to express CD86 and the high affinity Fc Receptor (CD64) (2:1 Treg:KT), which had been irradiated with 10,000 cGray and incubated with anti-CD3 (Orthoclone OKT3, Janssen-Cilag). In some experiments, Treg are stimulated with KT64/86 cells that are pre-loaded, irradiated, and frozen (1:1 Treg:KT). Irradiated feeder cells (2600 rads, CD8−/CD14−/CD19−/CD25−/CD3+) are added to CD3/28 bead cultures at 1:1 feeder:Treg. Tregs are cultured in X-Vivo-15 media (BioWhittaker) supplemented with 10% human AB serum (Valley Biomedical), GlutaMAX (Gibco) and N-acetylcysteine (USP). Recombinant IL-2 (300 IU/ml, Chiron) are added on day 2 and maintained for culture duration. Cultures are maintained at 0.3-0.5×106 viable NC/ml every 2-3 days.

In another example, to further expand the target cells, the above FACS-isolated disease related antigen specific cells isolated from leukocytes expanded with pMHC multimer are plated at ˜2.5×10⁵ cell per well in multiple wells of a 24-well plate (Nunc) and activated with Dynabeads CD3/CD28 CTS anti-CD3/anti-CD28-coated microbeads (Life Technologies) at a 1:1 bead/cell ratio. Cells are cultured either in X-VIVO 15 or in X-VIVO 15 supplemented with 10% human heat-inactivated pooled AB serum. At day 2, the culture volume is doubled and IL-2 was added (Proleukin, 300 IU/ml; Prometheus). Cells are resuspended, fresh medium and IL-2 are added at days 5, 7, 9, and 12, and the cells are transferred to cell culture plates and flasks (Nunc), and/or bags (Saint-Gobain) of increasing size to maintain a seeding density of ˜2×10⁵ to 3×10⁵ cells/ml in plates or flasks and a concentration of 500,000/ml in bags. On day 9, cells are re-stimulated with fresh anti-CD3/anti-CD28-coated beads at a 1:1 ratio. On day 14, cells are consolidated and de-beaded using a MaxSep magnet, and bead removal is verified via flow cytometry. Briefly, Dynabeads CD3/CD28 CTS (Invitrogen, catalog no. 402.03D) and Spherobeads (BD, catalog no. 556291) are used as controls for determining instrument settings and defining Dynabeads gate based on forward scatter (FSC) versus side scatter (SSC) followed by FL2 versus FL3 channels on FACSCalibur. Triplicate samples of expanded Tregs at ˜5×10⁶ cells/ml are analyzed, and a number of cells and Dynabeads in each sample are collected to determine cell number and bead number contained within each sample. The average bead count and average cell count are used to calculate the bead/cell ratio. The product is prepared as a cell suspension of fresh, non-cryopreserved cells in sterile infusion solution composed of 1:1 PlasmaLyte A/5% dextrose, 0.45% NaCl (Baxter) containing 0.5% human serum albumin (HSA) (Grifols), all supplied as U.S. Food and Drug Administration-approved drugs (PlasmaLyte A and dextrose/NaCl) or licensed products (HSA) for injection and conforming to U.S. Pharmacopeial Convention (USP) standards.

In another example, to further expand the target cells, the disease related antigen specific CD8+ Treg cells (e.g. those prepared above) are stimulated with CD3/28 beads at 1:5 ratio (one bead to 5 cells)+rhlL-2 (50 U/ml) CD8Medium or with TGF131 (5 ng/ml) CD8TGF13 in AIM-V serum-free medium containing Hepes buffer (10 mM), sodium pyruvate (1 mM), glutamine and penicillin and streptomycin in 24 or 48 well plates. On day 3, cells are split and fresh culture medium with IL-2 (30-50 U/ml) and is added to the wells. Additional IL-2 (50 U/ml) is added the day before harvest at day 5 or 6, and the beads are removed. In experiments to assess cytokine production, the CD8 cells are stimulated with PMA and Ionomycin for 6 hours. Brefeldin A is added one hour later and the cells are permeabilized (Fix and Perm Kit™ (BD) and stained for IL-2, IFN-gam, TNF-a and IL-17. Intracellular cytokine production is determined by flow cytometry.

In another example, the said isolated disease related antigen specific CD4+ cells at 10⁶/ml are stimulated in flat-bottom plates at a 1:1 bead-cell ratio with anti-CD3 and anti-CD28 or p31-I-Ag7mIgG2a and anti-CD28-coated beads in medium supplemented with 2000 IU/ml human rIL-2 (Chiron). Complete DMEM is used. Cultures are expanded with IL-2-supplemented medium when needed. Beads are removed at the end of the culture period before further experimentation. To remove coated latex beads, cultures are incubated with biotinylated anti-mouse IgG2a (Southern Biotechnology Associates) and biotinylated anti-hamster Ig (Vector Laboratories), washed, incubated with streptavidin microbeads (Miltenyi Biotec), washed, and run over an MS column (Miltenyi Biotec). Cells in the flow through fraction are collected. Next the purified cells are adoptive transferred to a subject in need.

In another example, the isolated disease related antigen specific CD8⁺ Tregs are seeded at 3×10⁵/ml in complete RPMI1640 medium 10% AB serum, IL-2 (1,000 U/ml) and IL-15 (10 ng/ml), coated anti-CD3 mAb (1 μg/ml), soluble anti-CD28 mAb (1 μg/ml), and/or allogeneic APCs at 1:4 Tregs:APCs ratio. At day 7, expanded cells are diluted at 1.5×10⁵/ml and stimulated again. IL-2 and IL-15 cytokines are freshly added at days 0, 7, 10, and 12. Cyclosporine A (CsA, 45 ng/ml), or rapamycin (45 ng/ml), or methylprednisolone (MPr, 500 pg/ml), or tacrolimus (2 ng/ml), or mycophenolate mofetil (MPA, 1 μg/ml) or their combination can be optionally added. For long-term expansion, Tregs are stimulated again with coated anti-CD3 (1 μg/ml), soluble anti-CD28 mAb (1 μg/ml) at days 14 and 21 and IL-2 and IL-15 cytokines are added every 2 days from days 7 to 28. Next the purified cells are adoptive transferred to a subject in need.

The current invention also discloses CAR-T or TCR-T and using it to treat autoimmune disease, allergy and to induce immune tolerance for specific antigens. The current invention discloses a method to treat an autoimmune disease, an allergy and to induce immune tolerance for an antigen with cytotoxic immune cell selected from CAR-T or TCR-T or engineered NK cell or engineered NKT cell or engineered macrophage such as CAR-macrophage. The method comprises the following steps: construct CAR-T or TCR-T or engineered NK cell or engineered NKT cells or engineered macrophage that can selectively bind with disease related peptide-MHC II complex, expand said cells in vitro to reach a desired number of target cells (e.g. 10⁶-10⁹ copies), and then infuse back the expanded immune cells to the subject for desired therapeutical effect. It can be either autologous transfer or allogeneic transfer. The method and protocol to build CAR-T or TCR-T and the resulting engineered T cells can be the same as those used for cancer therapy except the chimeric antibody receptor in CAR-T or gene modified TCR in TCR-T is engineered to target the disease related antigen peptide-MHC II complex of the subject in need instead of the tumor marker binding TCR or CAR of the current CAR-T or TCR-T for cancer treatment. Genetically engineered NK cell and NKT cell or genetically engineered macrophage can be engineered to express affinity ligand such as CAR or TCR receptor for disease related antigen peptide-MHC II complex of the subject to treat corresponding autoimmune diseases or induce immune intolerance. The resulting CAR-T or TCR-T or engineered NK cell or engineered macrophage will inactivate the APC that presents disease related antigen therefore inhibit the activation of effector T cells. The CAR-T or TCR-T or NK cell or macrophage can be engineered to express PD-L1 either as free protein or membrane bound form, which will enhance their immune inhibiting activity. The method and protocol of PD-L1 genetic overexpression can be adopted from well-known prior arts and publications such as those described in PD-L1 genetic overexpression or pharmacological restoration in hematopoietic stem and progenitor cells reverses autoimmune diabetes DOI: 10.1126/scitranslmed.aam7543. The CAR-T or TCR-T or NK cell or engineered macrophage can also be engineered to delete their PD-1 expression to avoid the interference from their expressed PD-L1. Other immune suppression cytokine can also be over expressed by genetic modification to these T cells or NK cells or NKT cells, such as IFN-gama, IL-10, IL-21, IL-35 and TGF beta, and optionally their endogenous receptors for these immune suppression cytokines can be deleted by genetic editing. Optionally introducing FOXP3 expression with retro virus can also be performed. When allogeneic cells are used, they can be engineered to not to express MHC on their surface and also to express KIR inhibitory ligand such as HLA-E, HLA-G with optional human cytomegalovirus (HCMV) glycoprotein UL40 or its fragment to reduce the host rejection. For example, for NOD mice expressing MHC II IAg7, CAR-T with chimeric antibody receptor against 2.5 mi/IAg7 pMHC or against IGRP4-22/IAg7 pMHC or against IGRP128-145/IAg7 pMHC or against their combinations are constructed; alternatively TCR-T with gene modified TCR against 2.5 mi/IAg7 pMHC or against IGRP4-22/IAg7 pMHC or against IGRP128-145/IAg7 pMHC are constructed; the resulting CAR-T or TCR-T are expanded to >10⁶ copies in vitro and then infused them back to the mice to treat its diabetes. In some preferred embodiments, the CAR-T or TCR-T only need to be constructed to against one pMHC instead of their mixtures of a dieses related pMHC. In another example, to treat Type I diabetes patient with MHCII type HLA-DRB1*0401/DRA, CAR-T or NKT with chimeric antibody receptor against GAD555-567-HLA-DRB1*0401/DRA pMHC or against IGRP23-35-HLA-DRB1*0401/DRA pMHC or against their combinations are constructed; alternatively TCR-T with gene modified TCR against GAD555-567-HLA-DRB1*0401/DRA pMHC or against IGRP23-35-HLA-DRB1*0401/DRA pMHC or against their combinations are constructed; the resulting CAR-T or NKT or TCR-T are expanded to >10⁸ copies in vitro and then infused them back to the patient to treat diabetes. The CAR-T or NKT or TCR-T in the above examples can be either autologous or allogeneic.

US patent application Ser. No. 16/271,877, 62/649,579 and PCT application PCT/US19/17405 by the current inventor disclosed genetically engineered oncolytic microbes (e.g. virus and bacterial) for cancer treatment. In some embodiments, the cancer cell killing/inhibiting microbes (e.g. virus and bacterial) can also be engineered to express or produce or secret immune activity enhancing agent with recombination technology either in active molecule form or prodrug form that can be converted to active form in tumor microenvironment. Suitable immune activity enhancing agent can be selected from TLR agonist such as bacterial lipoprotein including triacyl lipopeptides, bacterial peptidoglycans as TLR 2 agonist, lipoteichoic acid, zymosan (beta-glucan), heat shock proteins, bacterial flagellin, profilin and bacterial diacyl lipopeptides, TLR peptide/protein agonist disclosed in patent application serial numbers WO2018055060A1, WO2013120073A1, WO2016146143A1 and US20180133295A1, or their combinations. Suitable immune activity enhancing agent can also be selected from granulocyte macrophage colony-stimulating factor, immunostimulatory monoclonal antibody, antibody for CD137, FMS-like tyrosine kinase 3 ligand (FLT3L), T-cell-tropic chemokines such as CCL2, CCL1, CCL22 and CCL17; B-cell chemoattractant such as CXCL13, Interferon gamma, type I IFN (e.g. IFN-a, IFN-beta); tumor necrosis factor (TNF)-beta, TNF-alpha, IL-1, Interleukin-2, IL-12, IL-6, IL-24, IL-2, IL-18, IL-4, IL-5, IL-6, IL-9, IL-28B and IL-13 or their derivatives, CD1d ligand, Vα14/Vβ8.2 T cell receptor ligand, iNKT agonist, antibody against OX 40, tumor necrosis factor, interferon gamma (IFNγ), Treg inhibitory agent such as inhibitory antibody against Treg (such as antibody against CD4, CD25, FOXP3 and TGF-β or its receptor) or their combinations. Furthermore, in some embodiments, the cancer cell killing/inhibiting microbes (e.g. virus and bacterial) can also be engineered to express or produce or secret enzymes that can produce anti-cancer activity. Suitable enzyme can be selected from sialidase (e.g. bacterial sialidase such as V. cholerae sialidase or viral sialidase such as flu sialidase or animal sialidase or human sialidase), hyaluronidase (e.g. human recombinant Hylenex), adenosine deaminase (e.g. adenosine deaminase 2), peptide-N-glycosidase (e.g. PNGase F), b-N-acetylglucosaminidase (e.g. recombinant from Streptococcus pneumonia), other endo-β-N-acetylglucosaminidases (Endo D and Endo H), exoglycosidases (such as β-galactosidase, neuraminidase and N-acetyl-β-glucosaminidase) and enzymes that can degrade mucin's carbohydrate part, as well as collagenase such as those from bacterial or human MMP No. 1, No. 8, No. 13, and No. 18. Engineering bacterial or virus to express the protein/peptide or enzyme listed above can be done easily with recombinatant technology by a skilled in the art. There are many protocols and formats in prior publications that can be adapted for the current invention. For example, FIG. 13 in U.S. patent application Ser. No. 16/271,877 or PCT application PCT/US19/17405 showed an example of the construct of a JX-594 virus that can produce sialidase by replacing the GM-CSF sequence with a flu sialidase sequence in Pexa-Vec (JX-594) oncolytic virus. In another example, WO2018006005A1 disclosed pseudotyped oncolytic viral delivery of therapeutic polypeptides. It described pseudotyped oncolytic viruses comprising nucleic acids encoding an engager molecule. In some embodiments, the pseudotyped oncolytic viruses comprise nucleic acids encoding an engager molecule and one or more therapeutic molecules. The current invention can simply use the sequence or sequences of the said enzymes and or protein/peptide of the current invention (e.g. sialidase and/or IL-2) as the therapeutic molecules in the prior art pseudotyped oncolytic viruses to construct the virus desired by the current invention. WO2017132552A1 disclosed oncolytic viral vectors and uses thereof. One can use the vector design to express the desired protein/peptide/enzyme of the current invention in an oncolytic virus to be used in the current invention. Those protein/peptide/enzymes can also be easily incorporated into the plasmid of bacterial such as lactic acid bacterial to be expressed by it as shown in the FIG. 14 in U.S. patent application Ser. No. 16/271,877, which showed an anti-cancer bacterial that produce three desired proteins such as those listed above. For example, WO2016124239A1 disclosed recombinant probiotic bacteria for use in the treatment of a skin dysfunction, which express FGF2, IL4 and CSF1 by inserting nucleic acid sequence(s) encoding them. When these nucleic acid sequence(s) are replaced with nucleic acid sequence(s) of V. cholerae sialidase, collagenase clostridium histolyticum, adenosine deaminase 2 and N-acetyl-β-glucosaminidase, it becomes an embodiment of the previous invention.

In the current invention, similar to those described in US patent application Ser. No. 16/271,877, 62/649,579 and PCT application PCT/US19/17405 by the current inventor, these peptides, proteins and/or enzymes can be incorporated into engineered immune cells for cancer treatment such as T cell used in CAR-T or TCR-T or engineered NK, NKT cells or engineered macrophage. For example, an engineered T cell or NK, NKT cell or engineered macrophage for cancer immune therapy can be engineered to express human sialidase, adenosine deaminase 2 and collagenase either as secreted enzyme or membrane bound enzyme, as well as bacterial flagellin.

In some embodiments, the CAR-T or TCR-T or engineered NK, NKT cells or engineered macrophage can be engineered to express or produce or secret immune activity enhancing agent with recombination technology either in active molecule form or prodrug form that can be converted to active form in tumor microenvironment. Suitable immune activity enhancing agent can be selected from TLR agonist such as bacterial lipoprotein including triacyl lipopeptides, bacterial peptidoglycans as TLR 2 agonist, lipoteichoic acid, zymosan (beta-glucan), heat shock proteins, bacterial flagellin, profilin and bacterial diacyl lipopeptides, TLR peptide/protein agonist disclosed in patent application serial numbers WO2018055060A1, WO2013120073A1, WO2016146143A1 and US20180133295A1, or their combinations. Suitable immune activity enhancing agent can also be selected from granulocyte macrophage colony-stimulating factor, immunostimulatory monoclonal antibody, antibody for CD137, FMS-like tyrosine kinase 3 ligand (FLT3L), T-cell-tropic chemokines such as CCL2, CCL1, CCL22 and CCL17; B-cell chemoattractant such as CXCL13, Interferon gamma, type I IFN (e.g. IFN-a, IFN-beta); tumor necrosis factor (TNF)-beta, TNF-alpha, IL-1, Interleukin-2, IL-12, IL-6, IL-24, IL-2, IL-18, IL-4, IL-5, IL-6, IL-9, IL-28B and IL-13 or their derivatives, CD1d ligand, Vα14/Vβ8.2 T cell receptor ligand, iNKT agonist, antibody against OX 40, tumor necrosis factor, interferon gamma (IFNγ), Treg inhibitory agent such as inhibitory antibody against Treg (such as antibody against CD4, CD25, FOXP3 and TGF-β or its receptor) or their combinations. Furthermore, in some embodiments, the CAR-T or TCR-T or engineered NK, NKT cells can be engineered to express or produce or secret enzymes that can produce anti-cancer activity. Suitable enzyme can be selected from sialidase (e.g. bacterial sialidase such as V. cholerae sialidase or viral sialidase such as flu sialidase or animal sialidase or human sialidase), hyaluronidase (e.g. human recombinant Hylenex), adenosine deaminase (e.g. adenosine deaminase 2), peptide-N-glycosidase (e.g. PNGase F), b-N-acetylglucosaminidase (e.g. recombinant from Streptococcus pneumonia), other endo-β-N-acetylglucosaminidases (Endo D and Endo H), exoglycosidases (such as β-galactosidase, neuraminidase and N-acetyl-β-glucosaminidase) and enzymes that can degrade mucin's carbohydrate part, as well as collagenase such as those from bacterial or human MMP No. 1, No. 8, No. 13, and No. 18. The CAR-T or TCR-T or engineered NK, NKT cells can be engineered to express the above peptide or proteins only when they are activated, e.g. when they are in the tumor or bind with cancer cells. The expressed protein or peptide can be either in active molecule form or prodrug form that can be converted to active form in tumor microenvironment, for example, become active when the inactive prodrug form is cleaved by the peptidase or protease in the tumor, similar to the Probody therapeutics from Cytomx Therapeutics, XPAT from Amunix Inc or COBRAs from Maverick Therapeutics.

Engineering immune cells to express foreign proteins are well known to the skilled in the art and there are many prior arts can be readily adopted for the current invention, such as those described in Nature Biotechnology volume 36, pages 847-856 (2018). When allogeneic cells are used, they can be engineered to not to express MHC on their surface and also to express inhibitory KIR ligand such as HLA-E, HLA-G with optional human cytomegalovirus (HCMV) glycoprotein UL40 or its fragment to reduce the host rejection.

Compounds and compositions (e.g. the composition, conjugate, polymer and nano/micro particle disclosed in the current invention) described herein can be administered as a pharmaceutical or medicament formulated with a pharmaceutically acceptable carrier. Accordingly, the compounds may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration but may also be used for oral administration or contained in a metered dose inhaler or nebulizer for insufflation. Compounds may be formulated to include other medically useful drugs or biological agents. The compounds also may be administered in conjunction with the administration of other drugs or biological agents useful for the disease or condition to which the invention compounds are directed. The compound can be formulated in pharmaceutically acceptable carrier. As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the desired tissue or a tissue adjacent to the desired tissue. Pharmaceutically acceptable carriers are known to one having ordinary skill in the art may be used, including water or saline. As is known in the art, the components as well as their relative amounts are determined by the intended use and method of delivery. The compositions provided in accordance with the present disclosure are formulated as a solution for delivery into a patient in need thereof, and are, in some embodiments, focused on injection delivery.

Diluent or carriers employed in the compositions can be selected so that they do not diminish the desired effects of the composition. Examples of suitable compositions include aqueous solutions, for example, a saline solution, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents. In certain embodiments, a polymer matrix or polymeric material is employed as a pharmaceutically acceptable carrier. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, compositions provided herein may be formulated as films, gels, foams, or and other dosage forms. Suitable ionic strength modifying agents include, for example, glycerin, propylene glycol, mannitol, glucose, dextrose, sorbitol, sodium chloride, potassium chloride, and other electrolytes. Suitable pH buffering agents for use in the compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or IVIES. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8.

In some embodiments the said parenteral composition/formulation further include a viscosity enhancing agent to increase its viscosity before or after being injected, which acts as a sustained release formulation. In certain embodiments, the injection has a viscosity greater than 10,000 cps at room temperature. In certain embodiments, the injection has a viscosity greater than 100,000 cps at room temperature. In certain embodiments, the injection has a viscosity greater than 5,000,000 cps at room temperature. In certain embodiments, the injection has a viscosity of 11,000,000 cps at room temperature. Example of the viscosity enhancing agent can be found readily from known pharmaceutical acceptable excipient such as hyaluronic acid, starch and carbomer. In some embodiments, the viscosity enhancing agent is biodegradable. The injection formulation can also be a thermal phase changing formulation. Thermal phase changing formulation is a formulation that change its phase from liquid at low temperature (0-20° C.) or room temperature (25° C.) to semisolid/gel when temperature increases to close to body temperature (>30° C.) or body temperature (37° C.), which can use poloxamer as excipient. A thermal phase changing injectable formulation can be given as either subcutaneous injection or intramuscular injections or intradermal injections to induce antigen specific immune tolerance and treat corresponding autoimmune diseases or allergy. It has low viscosity at low or room temperature but high viscosity at body temperature. The preparation of this kind of high viscosity formulation and thermal phase changing injectable formulation can be adopted from related publications readily by the skilled in the art and are described previously in the current invention.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described. Dosage levels typically fall in the range of about 0.001 up to 10 mg/kg/day; with levels in the range of about 0.05 up to 5 mg/kg/day are generally applicable. A compound can be administered parenterally, such as intravascularly, intravenously, intraarterially, intramuscularly, subcutaneously, or the like. Administration can also be orally, nasally, rectally, transdermally or inhalationally via an aerosol. The compound may be administered as a bolus, or slowly infused. A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful initial doses in humans. Levels of drug in plasma may be measured, for example, by HPLC. The exact formulation, route of administration and dosage can be chosen by the individual physician in view of the patient's condition. In some embodiments, the compound is injected 1 mg/kg˜10 mg/kg to a subject in need either IV or SQ once a week for 2 months. In some embodiments, the compound is injected 1 mg/kg˜10 mg/kg either IV or SQ once per two weeks for 3 months.

In the current application, the “/” mark means “and” and/or “or” and/or their combination. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from textbooks such as the chemistry textbooks, scientific journal papers and other well-known reference sources.

1. A polymer conjugate to induce immune tolerance to an antigen comprising an antigen causing immune intolerance, an immunosuppressant, and a polysialic acid.

2. The conjugate according to claim 1, wherein the antigen is B cell antigen.

3. The conjugate according to claim 1, wherein the antigen is T cell antigen in MHC-peptide complex form.

4. The conjugate according to claim 1, wherein the immunosuppressant is selected from rapamycin, fujimycin and methotrexate.

5. The conjugate according to claim 1, wherein the antigen is double stranded DNA that can bind with autoantibody in systemic lupus erythematosus.

6. The conjugate according to claim 1, wherein the antigen is double stranded DNA and the immunosuppressant is rapamycin.

7. A composition to induce immune tolerance to an antigen comprising an antigen causing immune intolerance, an immunosuppressant and a viscosity enhancing agent.

8. The composition according to claim 7, wherein the immunosuppressant is rapamycin.

9. The composition according to claim 7, wherein the antigen is an allergen.

10. A formulation to induce immune tolerance to an antigen comprising an antigen causing immune intolerance, an immunosuppressant and thermal phase changing agent.

11. The composition according to claim 10, wherein the immunosuppressant is rapamycin.

12. The composition according to claim 10, wherein the antigen is an allergen.
